(19) United States
(12) Patent Application Publication
(10) Pub. No.: US 2026/0054022 A1
(43) Pub. Date: Feb. 26, 2026

(54) USING EAR-WORN DEVICES TO DETECT THERAPY DEVICES

(71) Applicant: ResMed Pty Ltd., Bella Vista (AU)

(72) Inventors: Liam Holley, Bella Vista (AU); Nicholas James MEALEY, Fort Lee, NJ (US)

(73) Assignee: ResMed Pty Ltd., Bella Vista (AU)

(21) Appl. No.: 19/304,781

(22) Filed: Aug. 20, 2025

Related U.S. Application Data
(60) Provisional application No. 63/685,325, filed on Aug. 21, 2024.
(60) Provisional application No. 63/685,324, filed on Aug. 21, 2024.
(60) Provisional application No. 63/685,322, filed on Aug. 21, 2024.
(60) Provisional application No. 63/813,243, filed on May 28, 2025.
(60) Provisional application No. 63/813,238, filed on May 28, 2025.
(60) Provisional application No. 63/813,222, filed on May 28, 2025.

(51) Int. Cl. A61M 16/00

(52) U.S. Cl. CPC A61M 16/024; A61M 16/0003; A61M 2205/15; A61M 2205/3303; A61M 2205/3306; A61M 2205/3327; A61M 2205/3331; A61M 2205/3375; A61M 2205/3576; A61M 2210/0662; A61M 2230/20

(57) ABSTRACT

A processor of an ear-worn device such as an earbud may generate a model of an ear canal based at least in part on a soundwave detected by two or more microphone arrays of the earbud. The processor may determine, based on a stored model of the ear canal and the model of the ear canal, a change of a geometry of the ear canal. The processor may determine, based on the change of the geometry of the ear canal, the soundwave and an oxygen saturation value of a bloodstream, a respiratory therapy device being used to treat a pathology. The processor may generate, based on the respiratory therapy device, a therapy recommendation. The recommendation may be outputted on one or more devices.

### CROSS-REFERENCE TO RELATED APPLICATIONS

This application claims the benefit of priority from U.S. Provisional Application No. 63/685,322, filed on Aug. 21, 2024, and from U.S. Provisional Application No. 63/685,324, filed on Aug. 21, 2024, and from U.S. Provisional Application No. 63/685,325, filed on Aug. 21, 2024, and from U.S. Provisional Application No. 63/813,222, filed on May 28, 2025, and from U.S. Provisional Application No. 63/813,238, filed on May 28, 2025, and from U.S. Provisional Application No. 63/813,243, filed on May 28, 2025, the entirety of each of which is hereby incorporated by reference.

### BACKGROUND

Patients are often prescribed respiratory therapy systems to treat pathologies such as obstructive sleep apnea (OSA). However, actual use of these systems is left to the individual patient. Therefore, it is difficult to determine whether the patient is actually using their prescribed systems. Furthermore, various elements of these systems may benefit from optimization. However, without sufficient data, these optimizations cannot be tailored to a particular patient.

### SUMMARY

Systems, methods, devices, non-transitory media, and apparatuses are disclosed for using ear-worn devices such as earbuds to detect therapy devices. In various embodiments, an earbud pair comprising two earbuds can be utilized to determine a patient's oxygen saturation level and generate a model of the ear canal based on soundwave data received by microphone arrays. This model can be used to detect changes in the geometry of the ear canal. Based on these changes, the earbud system can determine a respiratory therapy system being used by the patient to treat a pathology and generate a therapy recommendation.

The methods, systems, devices, and apparatuses described may be implemented to improve the functionality of a processor, such as a processor of a specific purpose computer, wearable device, respiratory monitor, and/or a respiratory therapy apparatus. Moreover, the described methods, systems, devices, and apparatus can provide improvements in the technological field of automated detection, management, monitoring, and/or treatment of respiratory conditions, including, for example, sleep disordered breathing.

### BRIEF DESCRIPTION OF THE SEVERAL VIEWS OF THE DRAWINGS

FIG. 1 illustrates a system that uses earbuds to detect therapy devices, in accordance with one embodiment.

FIG. 2 illustrates components of an earbud to detect therapy devices, in accordance with one embodiment.

FIG. 3A-FIG. 3B illustrate aspects of the subject matter in accordance with various embodiments.

FIG. 4 illustrates an aspect of the subject matter in accordance with one or more embodiments.

FIG. 5 shows a patient tracking device worn by a patient.

FIG. 6 shows a patient tracking device worn by a patient together with a patient interface.

FIG. 7 shows a view of the human ear canal.

FIG. 8 illustrates an aspect of the subject matter in accordance with one embodiment.

FIG. 9 illustrates an aspect of the subject matter in accordance with one embodiment.

FIG. 10 illustrates an aspect of the subject matter in accordance with one embodiment.

FIG. 11 illustrates a logic flow 1100 in accordance with one embodiment.

FIG. 12 illustrates a logic flow 1200 in accordance with one embodiment.

FIG. 13 illustrates a logic flow 1300 in accordance with one embodiment.

FIG. 14 shows a patient interface in the form of a nasal mask.

FIG. 15A-FIG. 15D illustrate components of a Respiratory Pressure Therapy (RPT) device.

FIG. 16 illustrates a computing system 1600 in accordance with one embodiment.

### DETAILED DESCRIPTION

Embodiments disclosed herein include techniques for using ear-worn devices to detect therapy devices being used by a patient, determine pathologies of the patient, predict pathologies of the patient, determine the position of sounds, track patient adherence to therapy, detect errors or configuration issues with therapy devices, and/or generate recommendations.

For example, embodiments disclosed herein include techniques for detecting respiratory therapy devices (also referred to as “respiratory therapy systems”) being used by a patient to treat pathologies (e.g., airway obstructions, etc.). In some embodiments, a respiratory therapy device is detected using an ear-worn device, such as one or more earbuds. For example, the ear-worn device may capture soundwaves using one or more microphone arrays, analyze the soundwaves, and determine the respiratory therapy device is being used based on the analysis. For example, the soundwaves may be compared to stored soundwaves and determine the sound is associated with a particular respiratory therapy device (e.g., a Continuous Positive Airway Pressure (CPAP) device, mandibular advancement device, or any type of intraoral therapy device). Similarly, the analysis may be based on the earbuds determining a location where the soundwave originated. For example, the ear-worn devices may determine a sound originated proximate to the mouth to determine an intraoral respiratory therapy device is being used.

In addition and/or alternatively, the use of the respiratory therapy device is detected based on using the ear-worn devices to monitor other physiological attributes of the patient. For example, if pulse oximeters in the earbuds determine the patient's oxygen saturation is increasing, embodiments disclosed herein may determine a respiratory therapy device is being used. Similarly, if the patient's oxygen saturation is decreasing, embodiments disclosed herein may determine the respiratory therapy device is not being used. In addition and/or alternatively, types of detected sounds may be used to determine whether a respiratory therapy device is being used. For example, the ear-worn devices may detect the sound of teeth grinding and determine an intraoral device is not being used.

In addition and/or alternatively, to detect the ear-worn devices may be used to generate a model of the geometry of one or both ear canals of the patient. For example, the ear-worn devices may emit sounds using one or more speakers and use the microphone arrays to detect reflections of the emitted sounds within the ear canal to determine the geometry of the ear canal. In addition and/or alternatively, the ear-worn devices may be used to actively monitor a patient to detect sounds and determine the geometry of the ear canal based on the detected sounds. The model of the geometry of the ear canal may be periodically determined and/or stored at a plurality of time intervals. Doing so allows any two models and/or geometries to be compared to determine changes in the geometry of the ear canal. Furthermore, over time, one or more stored models of the ear canal may be associated with a particular respiratory therapy device (or devices), pathology, and/or physiological attributes. Doing so allows a given model to be compared to stored models to identify a particular respiratory therapy device and/or pathology. More generally, the ear-worn devices may use the models to detect pathologies, e.g., based on stored models of the ear canal under various conditions (e.g., with an airway obstruction, etc.).

In addition and/or alternatively, the ear-worn devices may determine attributes of a detected device, e.g., cushions used in a specific type of mask, a hose used in a CPAP machine, etc. In some embodiments, the ear-worn devices may further determine errors or other problems with respiratory therapy systems and/or components thereof. For example, air leaks, improperly sized masks, and/or suboptimal configurations may be detected.

In some embodiments, the ear-worn devices may record and monitor the use of different devices by the patient to determine whether the patient is adhering to their prescribed therapy. For example, by periodically searching for and detecting respiratory therapy devices worn or otherwise used by a patient (e.g., at predetermined time intervals), the ear-worn devices may determine use patterns by the patient for each respiratory therapy device. Furthermore, by collecting physiological data, changes in the physiological data may be correlated with use and/or lack of use of a given respiratory therapy device. For example, average (or mean) oxygen saturation values may be computed during times the patient is using a respiratory therapy device and when the patient is not using the respiratory therapy device.

In some embodiments, the ear-worn devices may generate a recommendation. For example, by detecting sounds associated with air leaks (e.g., in a mask, conduit, etc.), embodiments disclosed herein may make recommendations to use a new type of mask, order replacement parts, or present instructions related to a detected type of respiratory therapy device. For example, when a leak is detected in a mask, replacement parts may be automatically ordered, better fitting mask types may be recommended, or the user manual for the specific mask being used may be automatically displayed on the patient's computing device (e.g., to assist the user to wear the mask properly, etc.). As another example, the recommendation may include adjusting the titration of a CPAP device, modifying the coefficients used in quadratic relationships between pressure drops and air flow in the CPAP device, causing the patient to move from a back-sleeping position to a side-sleeping position, etc. As another example, to improve patient adherence to therapy, the ear-worn devices may output a notification to encourage the patient to use their respiratory therapy devices. Such notifications may include visualizations of how often the devices are used, changes in physiological data when the devices are used/not used, etc. The notification may further be outputted via the ear-worn devices, a smartphone of the user, wearable devices of the user, etc.

In some embodiments, the ear-worn devices may detect pathologies. For example, the ear-worn devices may include a plurality of microphone arrays to detect sounds and one or more speakers to emit sounds. The ear-worn devices may analyze the sounds and determine a location of the sounds in the patient, e.g., using a position determination algorithm. For example, by detecting snoring sounds, obstructive sleep apnea (OSA) may be detected. The ear-worn devices may further determine that the sounds are associated with a respiratory therapy device, such as a mandibular advancement device used to treat an airway obstruction. In some embodiments, the ear-worn devices determine other attributes of the pathology, such as a type of the pathology (e.g., obstructive sleep apnea (OSA), a degree of the pathology (e.g., partial collapse, complete collapse, etc.)).

In some embodiments, hearing loss may be detected (e.g., emitting sounds to detect a reaction, monitoring the sounds generated by the ear over time to detect hearing loss, etc.). As another example, heart attack and/or stroke may be predicted based on localized turbulence sounds generated by damaged blood vessels. For example, by analyzing the startle response of a patient using earbuds, the earbuds may determine that the patient has hearing loss (e.g., where a sound does not startle or otherwise generate a response in the patient).

When the ear-worn devices detect a respiratory therapy device and/or a pathology in the patient, the ear-worn devices may cause any number and type of operations to be performed to indicate (e.g., a notification), diagnose, and/or treat the pathology. For example, the ear-worn devices may emit alarms, notifications, sounds, music, etc., to wake the patient, cause the patient to change sleeping positions, notify the patient, etc. Similarly, the ear-worn devices may transmit a notification to a device of the patient, a device of the patient's medical provider, etc. Further still, the ear-worn devices may cause one or more respiratory therapy devices to adjust one or more operating parameters to modify a therapy delivered by the respiratory therapy devices to the patient. Embodiments are not limited in these contexts.

Advantageously, embodiments disclosed herein provide techniques to identify a range of respiratory therapy devices being used to treat one or more pathologies and effect a treatment or prophylaxis for the pathology. By leveraging sensors integrated into ear-worn devices that can identify different devices and/or pathologies, more devices and pathologies can be identified and treated over time at a reduced cost to the patient. Furthermore, doing so allows the detection and treatment of pathologies that would otherwise go undetected and/or untreated. Further still, by identifying a particular device to treat pathology, embodiments disclosed herein may determine a particular recommendation, e.g., a treatment for the pathology, a modified parameter for the device, a modified configuration of the device, reordering supplies for the device, detecting problems associated with the device and generating recommendations to correct the problems, etc. For example, an ear-worn device may instruct a device to change titration delivered to the patient, change an algorithm used by the device to deliver positive pressure to the entrance of the airways of the patient, detect an error with a device and order a replacement part, recommend new devices, etc. Embodiments are not limited in these contexts.

Aspects of the present disclosure and certain features, advantages, and details thereof are explained more fully below with reference to the non-limiting examples illustrated in the accompanying drawings. Descriptions of well-known processing techniques, systems, components, etc. are omitted to not unnecessarily obscure the disclosure in detail. The detailed description and the specific examples, while indicating aspects of the disclosure, are given by way of illustration only, and not by way of limitation. Various substitutions, modifications, additions, and/or arrangements, within the spirit and/or scope of the disclosed aspects will be apparent to those skilled in the art from this disclosure. Note further that numerous aspects and features are disclosed herein, and unless inconsistent, each disclosed aspect or feature is combinable with any other disclosed aspect or feature as desired for a particular embodiment of the concepts disclosed herein.

Unless described or implied as exclusive alternatives, features throughout the drawings and descriptions should be taken as cumulative, such that features expressly associated with some particular embodiments can be combined with other embodiments. Like numbers refer to like elements throughout.

While certain exemplary embodiments have been described and shown in the accompanying drawings, it is to be understood that such embodiments are merely illustrative of, and not restrictive on, the broad disclosure, and that this disclosure not be limited to the specific constructions and arrangements shown and described, since various other changes, combinations, omissions, modifications and substitutions, in addition to those set forth in the above paragraphs, are possible. Those skilled in the art will appreciate that various adaptations, modifications, and combinations of the herein described embodiments can be configured without departing from the scope and spirit of the disclosure. Therefore, it is to be understood that, within the scope of the included claims, the disclosure may be practiced other than as specifically described herein.

Additionally, illustrative embodiments are described below using specific code, designs, architectures, protocols, layouts, schematics, or tools only as examples, and not by way of limitation. Furthermore, the illustrative embodiments are described in certain instances using particular software, tools, or data processing environments only as example for clarity of description. The illustrative embodiments can be used in conjunction with other comparable or similarly purposed structures, systems, applications, or architectures. One or more aspects of an illustrative embodiment can be implemented in hardware, software, or a combination thereof.

As understood by one skilled in the art, program code, as referred to in this application, can include both software and hardware. For example, program code in certain embodiments of the present disclosure can include fixed function hardware, while other embodiments can utilize a software-based implementation of the functionality described. Certain embodiments combine both types of program code.

The terms “coupled,” “fixed,” “attached to,” “communicatively coupled to,” “operatively coupled to,” and the like refer to both (i) direct connecting, coupling, fixing, attaching, communicatively coupling; and (ii) indirect connecting coupling, fixing, attaching, communicatively coupling via one or more intermediate components or features, unless otherwise specified herein. “Communicatively coupled to” and “operatively coupled to” can refer to physically and/or electrically related components.

Some of the figures may include a logic flow. Although such figures presented herein may include a particular logic flow, it can be appreciated that the logic flow merely provides an example of how the general functionality as described herein can be implemented. Further, a given logic flow does not necessarily have to be executed in the order presented unless otherwise indicated. Moreover, not all acts illustrated in a logic flow may be required in some embodiments. In addition, the given logic flow may be implemented by a hardware element, a software element executed by a processor, or any combination thereof. The embodiments are not limited in this context.

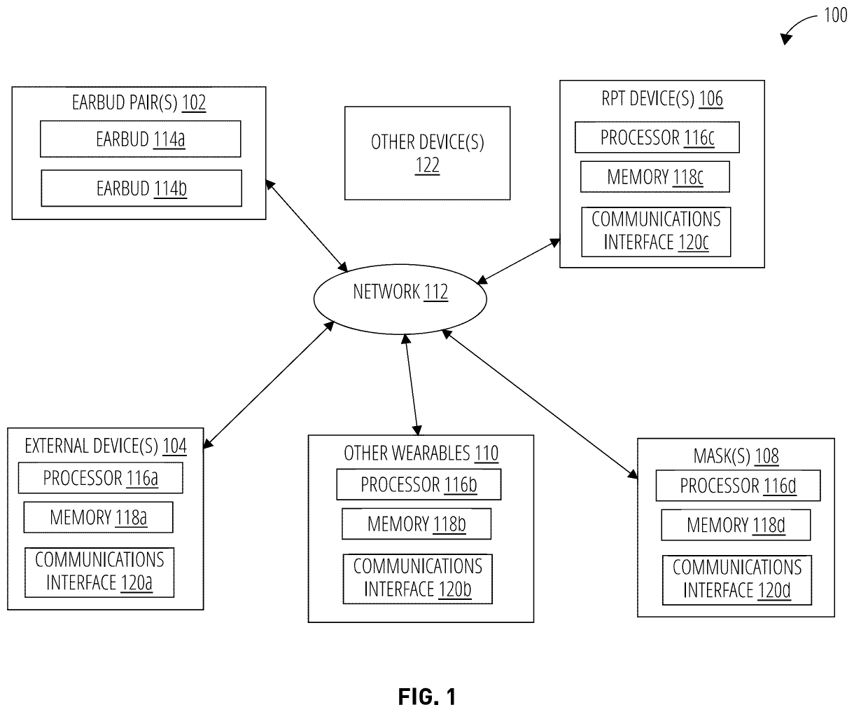

FIG. 1 illustrates a system 100 in accordance with one embodiment. The system 100 may be a system that uses earbuds to detect therapy devices being used by a human patient (not pictured). Therefore, one or more components of the system 100 may be part of a patient health system. Embodiments are not limited in these contexts.

As shown, the system 100 includes one or more earbud pairs 102, one or more external devices 104, one or more respiratory therapy (RPT) devices 106, one or more masks 108, and one or more other wearables 110 communicably coupled via a communications network 112. In some embodiments, the system 100 includes one or more other therapy devices 122, e.g., intraoral therapy devices, mouth tape, teeth guards, nerve stimulation devices (e.g., transcutaneous electrical nerve stimulation (TENS) devices, percutaneous electrical nerve stimulation devices etc.), devices that stimulate the motion of the tongue, maskless respiratory therapy devices, or any other therapy device.

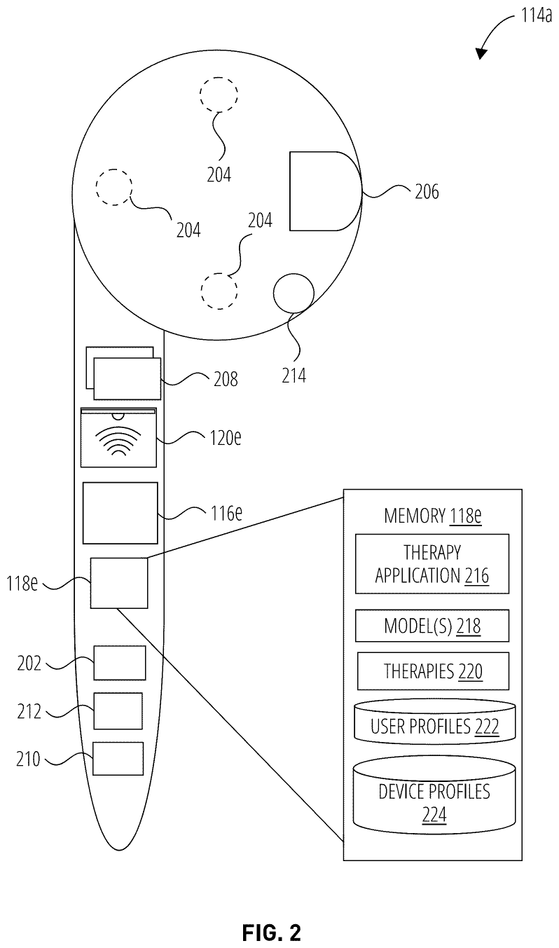

The earbud pair 102 includes an earbud 114a and an earbud 114b. Additional components of the earbuds 114a-114b are depicted in FIG. 2. Generally, earbuds 114a-114b are worn in, around, or proximate to the ear of a person. Although the “earbud” is used as one reference example herein, the disclosure is equally applicable to other types of ear-worn electronic devices. Therefore, embodiments are not limited to the earbud form factor.

The external devices 104 represent any type of device, such as a computing device, smartphone, laptop, tablet, hub, smart home device, medical provider device or system, medical device, networking device, Internet of things (IoT) device, and the like. The other wearables 110 represent any type of wearable device, such as smart watches, smart rings, smart goggles, smart glasses, medical devices, straps, and the like.

The RPT device 106 represents any respiratory therapy device or system, such as a Continuous Positive Airway Pressure (CPAP) device. More generally, the RPT device 106 is configured to generate a flow of air for delivery to the human airways via an interface such as a mask 108. In some embodiments, the RPT devices 106 include RPT devices that are implanted at least partially within the body of a patient.

As shown in FIG. 1, the external devices 104, RPT devices 106, masks 108, and other wearables 110 include a processor 116a, a processor 116b, a processor 116c, and a processor 116d, respectively. As shown in FIG. 2, the earbud 114a, which represents earbud 114b, similarly includes a processor 116e, a memory 118e, and a communications interface 120e.

The processors 116a-116e represent any type of processor circuit. Examples of processor circuits include an Intel® x86 processor, an ARM® processor, a 32-bit RISC CPU, a 16-bit RISC CPU, AMD® processors, and similar processors. Similarly, the external devices 104, RPT devices 106, masks 108, and other wearables 110 include a memory 118a, a memory 118b, a memory 118c, and a memory 118d, respectively. The memories 118a-118e represent any type of computer memory, such as volatile memory or non-volatile memory. To communicate via the network 112, the external devices 104, RPT devices 106, masks 108, and other wearables 110 include a communications interface 120a, a communications interface 120b, a communications interface 120c, and a communications interface 120d, respectively. The communications interfaces 120a-120e represent any type of data communications interface, such as a wireless (or wired) transceiver. In some embodiments, the other therapy devices 122 similarly include a processor, memory, and communications interface to the network 112.

The network 112 may be any type of data communications network. In some embodiments, the network 112 is a wireless communications network. Examples of wireless communications networks include an IEEE 802.11 wireless network, Wi-Fi, Bluetooth®, Bluetooth Low Energy (BLE), near-field communication (NFC), radio frequency identification (RFID), radio frequency (RF) networks, or any other type of wireless communication network. Therefore, the communications interfaces 120a-120e are configured to support IEEE 802.11 wireless networks, Wi-Fi, Bluetooth, Bluetooth Low Energy (BLE), near-field communication (NFC), radio frequency identification (RFID), radio frequency (RF) networks, or any other type of wireless communication network. Furthermore, the network 112 represents direct wireless communications between the entities of the system 100.

Collectively, the components of the system 100 (or any subset thereof) are configured to monitor a human patient, collect data from the patient, deliver therapy (e.g., a treatment) to the patient, detect therapies delivered to the patient, detect respiratory therapy systems (and/or components thereof) used to deliver therapies to the patient, modify therapies delivered to the patient, and/or generate recommendations. For example, the earbuds 114a-114b may collect data from the patient, detect a device used to treat a pathology, and send an alert via the network 112. The collected data may include data from the external devices 104, other wearables 110, masks 108, and/or RPT devices 106. For example, the alert may include an indication of the detected device, an error with the device, and a recommendation for the patient (e.g., to correct the error). For example, the alert may be sent to an external device 104, providing the alert to a medical provider system. Doing so allows the medical provider to prescribe a treatment, e.g., to prescribe a different type of mask, change titrations, change therapy devices, correct errors, etc. In another example, the alert may be sent as an indication to the RPT device 106 and/or the mask 108, which may modify the type of therapy, attributes of the therapy, and/or a duration of the therapy provided to the patient. In some embodiments, the recommendation is outputted by other devices of the patient, e.g., smartphones, smartwatches, etc. Stated differently, any recommendation, notification, or other content disclosed herein may be outputted via the external devices 104, RPT devices 106, other wearables 110 and/or masks 108. Embodiments are not limited in these contexts.

FIG. 2 illustrates an example earbud 114a that can communicatively couple with earbud 114b to form a pair of untethered, wireless earbuds according to some embodiments of the present technology. Although earbud 114a is depicted, earbud 114b includes the components depicted in FIG. 2.

The earbud 114a uses communications interface 120e to communicatively couple with another wireless earbud, e.g., earbud 114b, and to pair with a source device, e.g., a companion communication device (e.g., external devices 104 such as smartphones) that can provide audio data that the earbuds 114a can reproduce as audio signals for a user of the earbuds 114a, 114b. In some embodiments, a process of pairing the earbuds 114a, 114b is initiated when the earbuds 114a, 114b are contained within a housing/case, not pictured for clarity. In some circumstances, once a pairing mode is enabled for the earbuds 114a, 114b, the earbuds 114a, 114b remain in the enabled pairing mode until one or more of the following occurs: (i) the earbud 114a or 114b pairs with a companion communication device, (ii) a pairing mode of the earbuds 114a, 114b times out (e.g., the earbud 114a or 114b does not pair with a companion communication device within a fixed time period, such as thirty seconds), (iii) the earbud 114a or 114b is removed from the case, (iv) the wireless earbud case commands one or more both of the earbuds 114a, 114b to exit the pairing mode, or (v) the companion communication device commands the earbuds 114a, 114b to exit the pairing mode. The earbud 114a can also include a battery 202 and sensors 204 for detecting a wearing status of the earbud 114a, e.g., when the earbud 114a is placed in and/or removed from an ear, whether the earbud 114a is in a user's ear, e.g., an in-ear wearing status, or is not in a user's ear, e.g., an out-of-ear wearing status.

Additionally, the earbud 114a includes an audio output device such as a speaker 206 for converting a received signal, e.g., which can include audio data, into audible sound. The signal can be received from a paired companion communication device via the communications interface 120e. The memory 118e in the earbud 114a stores firmware for operating the earbud 114a as well as data for coupling with other wireless earbuds and for pairing the earbud 114a with companion communication devices. For example, the memory 118e in the earbud 114a can store a connection history for companion communication devices with which the earbud 114a has previously paired. The connection history can include data for automatically pairing the earbud 114a with the companion communication device without having to configure a connection between the earbud 114a and the companion communication device (e.g., enter a password, exchange shared secrets, etc.). For example, the connection history can include one or more link keys for connecting to a wireless network such as network 112 (e.g., Bluetooth link keys). The memory 118e of the earbud 114a can also store a MAC address that uniquely identifies the earbud 114a as well as store a paired partner MAC address of another wireless earbud 114b that has previously coupled with the earbud 114a. The memory 118e also stores instructions that, when executed by the processor, causes the earbud 114a to communicatively couple with another wireless earbud.

As shown, the earbud 114a includes one or more sensors 204, one or more speakers 206, two or more microphone arrays 208, a haptic feedback module 210, an accelerometer 212, and a pulse oximeter 214. The speakers 206 are devices to output audio, e.g., soundwaves. Each of the microphone arrays 208 includes a plurality of microphones (not pictured) that are configured to detect and record audio data, e.g., soundwaves. Therefore, a given earbud 114a, 114b, may include a plurality of microphone arrays 208, with each microphone array 208 including a plurality of microphones. The total number of microphones in each earbud 114a, earbud 114b may, therefore, number in the tens, hundreds, thousands, or more. In some embodiments, a first one of the microphone arrays 208 is located at a first end of the earbud 114a (e.g., nearest to the ear canal), while a second one of the microphone arrays 208 is located at an opposite end of the earbud 114a (e.g., farthest from the ear canal). In such embodiments, one or more other microphone arrays 208 may be located between the first and second microphone arrays 208. Embodiments are not limited in these contexts.

The haptic feedback module 210 is a device that generates vibrations or other tactile sensations, such as piezoelectric actuators/sensors, etc. The haptic feedback module 210 may detect reflections thereof, e.g., reflections of vibrations from the ear canal when the earbud 114a is worn by a patient, which may be useful in detecting a pathology in the patient or detecting a therapy device used by the patient. The accelerometer 212 is a device that measures the rate of change of velocity (e.g., acceleration) of the earbud 114a along one or more axes, providing data on movement and orientation. For example, data from the accelerometer 212 may provide information on the orientation of a human head, body, etc., which may be useful when the therapy application 216 processes data to detect a pathology in the patient and/or detect a therapy device used or otherwise worn by the patient. The pulse oximeter 214 is a peripheral oxygen saturation sensor that is configured to determine a peripheral oxygen saturation (SpO₂) value of a bloodstream of a patient. Stated differently, the pulse oximeter 214 is configured to detect the oxygen levels of a patient.

The sensors 204 represent any type of sensor, such as a pressure sensor, a flow rate sensor, a temperature sensor, a motion sensor, a camera, an infrared (IR) sensor, a photoplethysmogram (PPG) sensor, an electrocardiogram (ECG) sensor, an electroencephalography (EEG) sensor (e.g., an electrode), a capacitive sensor, an electromyography (EMG) sensor, an oxygen sensor, an analyte sensor, a moisture sensor, a light detection and ranging (LiDAR) sensor, an electrooculography (EOG) sensor, a galvanic skin response (GSR) sensor, or a carbon dioxide (CO2) sensor.

As shown, the memory 118e of the earbud 114a includes a therapy application 216, one or more models 218, a data store of therapies 220, a data store of user profiles 222, and a data store of device profiles 224. The therapy application 216 is generally configured to detect pathologies in a patient, detect therapy devices worn or otherwise used by a patient, generate models of the ear canal of the patient, predict pathologies of the patient, determine the position of sounds, track patient adherence to therapy, detect errors or configuration issues with therapy devices, and/or generate recommendations.

The user profiles 222 store a plurality of attributes for a plurality of users. For example, the user profiles 222 may store data recorded by the sensors 204 (e.g., oxygen saturation values, respiratory rate, etc.), pathologies associated with the user, devices used by or otherwise prescribed to the user (e.g., RPT device 106, a mask 108, etc.), models of the ear canal of the user, use of the devices prescribed to the user (e.g., a log of entries detailing dates and times when a patient uses their mask 108, RPT device 106, etc.), or any other attribute of the user. The device profiles 224 include data describing different devices, such as RPT devices 106, masks 108, other wearables 110, other therapy devices 122, external devices 104, etc. Example attributes stored in the device profiles 224 include device type, device model, device function, sound profiles, how the device is worn or otherwise used by the patient, configurations, associated components, and the like. The device profiles 224 may further include associations between devices (e.g., RPT devices 106, masks 108, other wearables 110, other therapy devices 122, etc.) and one or more pathologies for which the devices are prescribed to provide therapy.

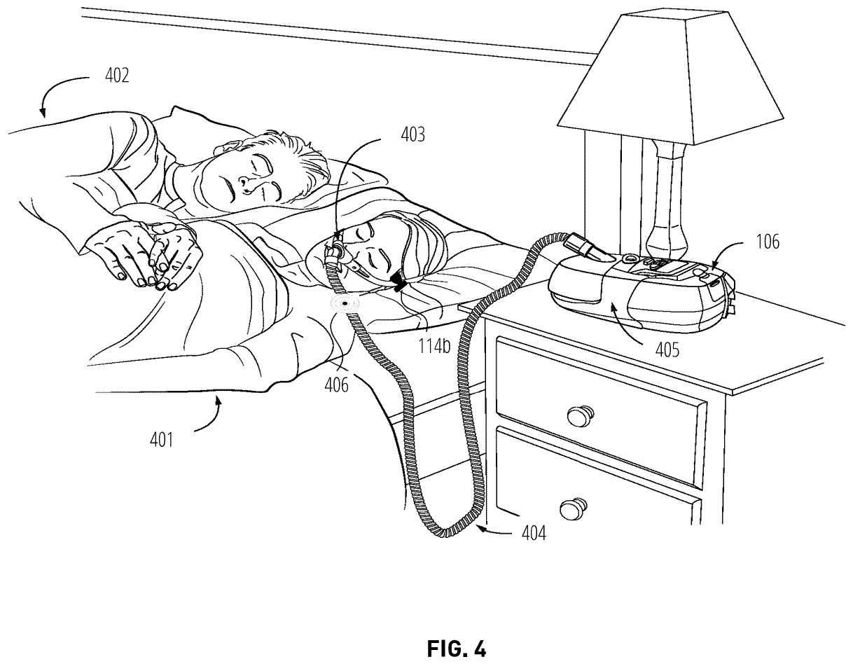

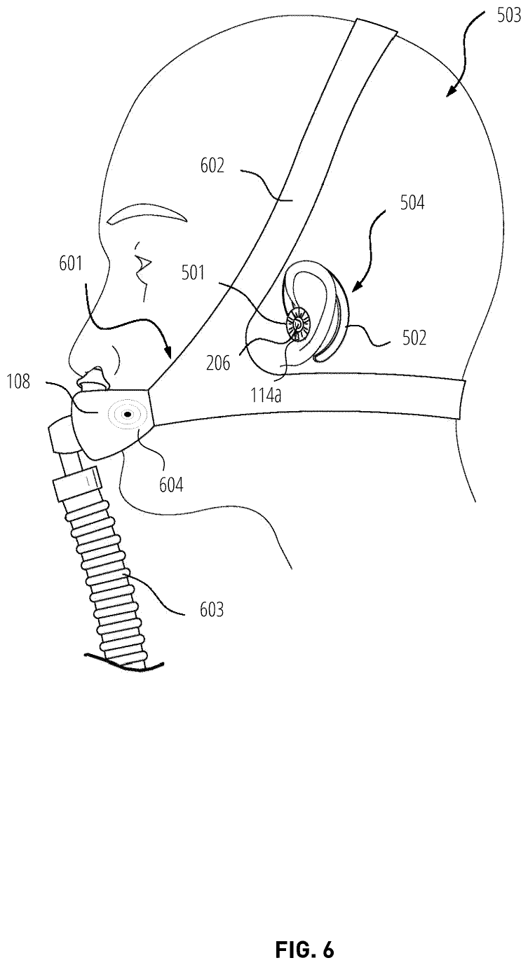

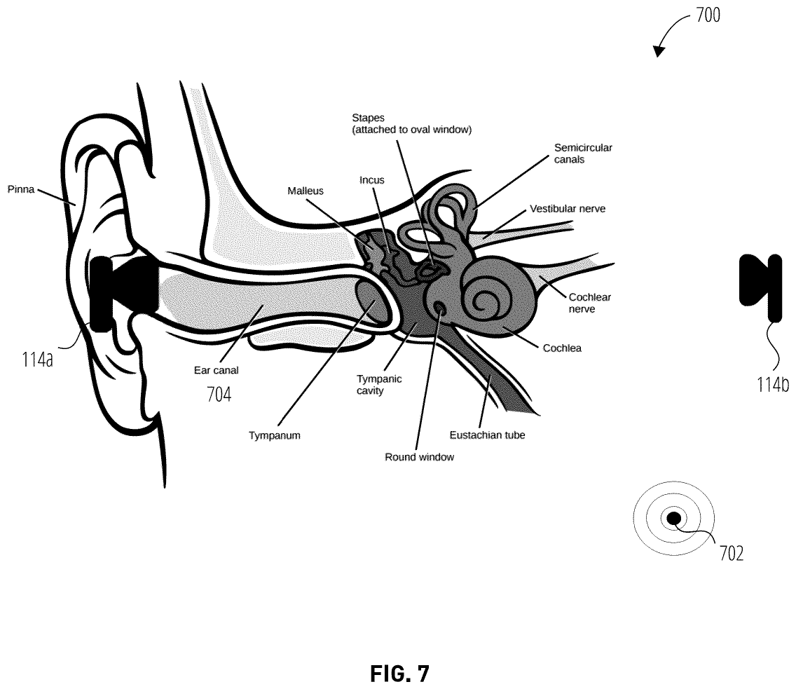

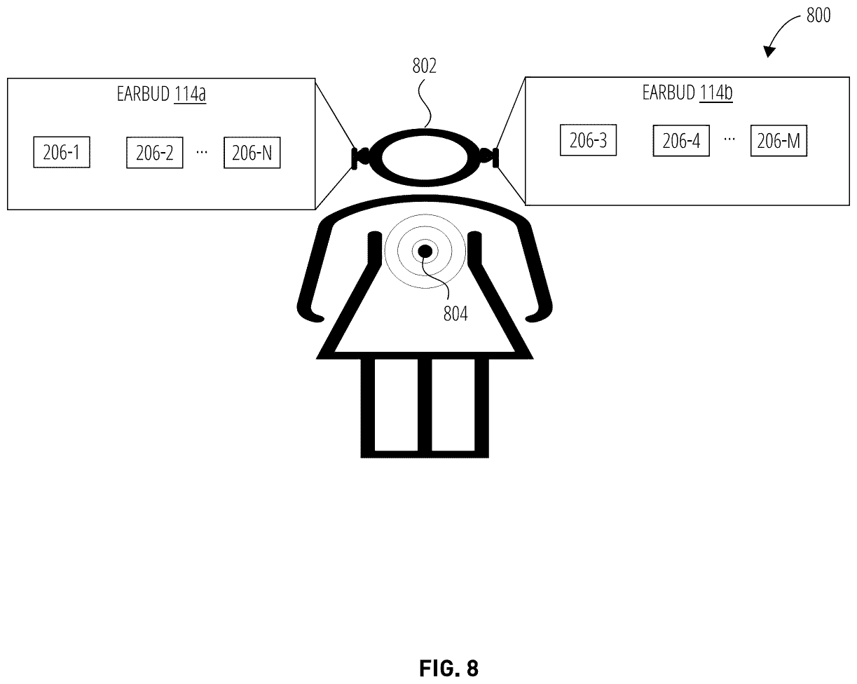

In some embodiments, the therapy application 216 uses sound to detect therapy devices used by the patient. Examples of such sounds include soundwaves 406, 604, 702, and 804, of FIG. 4, FIG. 6, FIG. 7, and FIG. 8, respectively. In some embodiments, the sounds may be generated by the therapy device. In some embodiments, the soundwaves are detected by the earbud pair 102 and used to generate a model of the ear canal. In some embodiments, at least a portion of the detected soundwaves are reflections of vibrations or other sounds emitted into the ear canal by the speakers 206 and/or haptic feedback modules 210.

In some embodiments, the therapy application 216 uses additional information collected by the sensors 204 to determine that a patient is wearing or otherwise using a therapy device. For example, the pulse oximeter 214 may record oxygen saturation (SpO₂) values from the bloodstream of the patient at predetermined intervals (and/or based on instructions from the therapy application 216, e.g., when the therapy application 216 detects a sound, detects a therapy device, detects a pathology, etc.). In some embodiments, the oxygen saturation values are stored in the user profile 222 of the patient. The therapy application 216 may use the oxygen saturation values to determine, at least in part, whether the patient is using a therapy device. For example, if the patient's oxygen saturation is below a threshold, the therapy application 216 may determine that the patient is not using an RPT device 106 (e.g., because the patient is not experiencing suitable SpO₂ levels). As another example, if the oxygen saturation is above a threshold, the therapy application 216 may determine that the patient is using an RPT device 106. The thresholds may be any type of threshold, such as predetermined minimum/maximum oxygen saturation thresholds, thresholds that are associated with a specific patient (e.g., patient's average oxygen saturation while using an RPT device 106 and/or average oxygen saturation while not using an RPT device 106, etc.), thresholds associated with a group of patients (e.g., patients with a specific type of pathology, etc.). In some embodiments, the thresholds are stored in a patient's user profile 222.

As another example, the sensors 204 may detect electrical signals to determine the patient is being treated with electrical stimulation. For example, an EEG sensor may detect electrical signals (e.g., crosstalk) from electrical stimulation devices applying electrical stimulation to the patient's body e.g., nerve stimulation (transcutaneous electrical nerve stimulation (TENS), percutaneous electrical nerve stimulation), electrical stimulation (e-stim) for physical therapy, etc.

In some embodiments, the therapy application 216 may use the position information from the accelerometer 212 and/or one or more other wearables 110 at least in part to detect the use of therapy devices by a patient. For example, if the accelerometer 212 and/or one or more other wearables 110 provide position information indicating the patient is lying on their stomach, the therapy application 216 may exclude certain types of face-worn therapy devices from consideration. Similarly, however, if the accelerometer 212 and/or one or more other wearables 110 provide position information indicating the patient is lying on their back, the therapy application 216 may consider certain types of face-worn therapy devices as possibly being worn by the patient. As another example, if the accelerometer 212 and/or one or more other wearables 110 provide position information indicating the patient is moving, the therapy application 216 may exclude static therapy devices from consideration (e.g., a user who is walking is likely not wearing a fixed RPT device 106).

The therapy application 216 and/or models 218 may generally use any location (or position) determination algorithm to determine the location where a sound detected by the microphone arrays 208 originated. Because multiple microphone arrays 208 are included in an earbud pair 102 (whether in a single earbud 114a, 114b or across both earbuds 114a, 114b), positions may be determined using measurements from these fixed points to compute the precise location a sound originated, e.g., using triangulation, trilateration, beamforming, single or multiple microphone acoustic impedance measurements, impulse/frequency response function measurements, etc.

For example, when the plurality of microphone arrays 208 detect a sound, the therapy application 216 and/or the models 218 may receive indications of the sounds (e.g., waveforms) from the microphone arrays 208. The therapy application 216 and/or the models 218 may determine a position of the sound source by measuring the time differences of arrival (TDOA) of the sounds experienced by the microphone arrays 208. As another example, the therapy application 216 and/or the models 218 may perform a mathematical cross-correlation operation that measures the similarity between two detected signals as a function of the time-lag applied to one of them. By shifting one signal in time and calculating the correlation at each shift, the cross-correlation allows the therapy application 216 and/or the models 218 to identify the time offset that maximizes the similarity between the two signals. Although discussed with reference to the therapy application 216 and/or the models 218, the microphone arrays 208 may include logic to perform the position and/or location determination described herein. Similarly, the external devices 104 may include instances of the therapy application 216, models 218, therapies 220, and user profiles 222, e.g., to perform the processing described herein (e.g., based on receiving data collected by the earbuds 114a, 114b).

Furthermore, the microphone arrays 208, the processor 116e, the therapy application 216, and/or the models 218 may analyze the sounds to identify one or more attributes of the detected sounds (e.g., pressure, amplitude, wavelength, and/or frequency). Doing so may be useful to identify the sounds (and/or causes thereof), e.g., sounds associated with therapy devices, sounds associated with pathologies, airway obstructions, etc., as described herein. For example, the pressure, amplitude, wavelength, and/or frequency may be compared to one or more known sounds, e.g., to identify a known sound that is similar to the detected sound. As stated, the known sounds may be stored or otherwise reflected in the models 218 and/or the device profiles 224. Similarly, the models 218 may consider the attributes of the sound and return a known sound as being similar to the detected sounds. The known sounds may be stored by the therapy application 216, e.g., in the device profiles 224. Similarly, the known sounds and/or attributes thereof (e.g., pressure, amplitude, wavelength, frequency, etc.) may be stored as features in the models 218. Doing so allows the therapy application 216 and/or models 218 to match a detected sound to a known sound (e.g., based on one or more of pressure, amplitude, wavelength, frequency, etc.). For example, the therapy application 216 may analyze a sound and determine the sound is associated with a specific type of therapy device in the device profiles 224, e.g., an RPT device 106, a mask 108, other therapy devices 122, etc. In some embodiments, the therapy application 216 further considers data in the user profiles 222 to determine that a patient is using a therapy device (e.g., to identify a type of a therapy device prescribed to the patient, etc.).

In some embodiments, a device may be associated as a treatment for one or more pathologies in the device profiles 224. As such, the therapy application 216 and/or models 218 may identify a pathology associated with a detected device and/or sound. For example, the therapy application 216 and/or models 218 may receive a detected sound as input (including any attributes thereof). The therapy application 216 and/or models 218 may identify a known sound, such as the sounds made by an RPT device 106 that are stored in the device profiles 224 and/or models 218, and determine a patient is using the RPT device 106. The therapy application 216 and/or models 218 may identify a corresponding pathology based on the detected device, e.g., RPT device 106. The therapy application 216 and/or models 218 may further identify a therapy 220 associated with the detected device and/or pathology, such as changing one or more parameters of the RPT device 106 providing therapy to the patient.

In some embodiments, distances between respective pairs of the microphone arrays 208 in a given earbud 114a are stored in the memory 118e (e.g., in the therapy application 216, the models 218, etc.) to facilitate the detection of pathologies in a patient, e.g., for use in a location detection algorithm such as triangulation, beamforming, trilateration, single or multiple microphone acoustic impedance measurements, impulse/frequency response function measurements, etc. Similarly, the distance between earbud 114a and earbud 114b may be determined at predetermined time intervals such that both earbuds 114a, 114b, can be used to detect sounds (e.g., associated with devices worn by the patient and/or pathologies of the patient) over time. For example, the processor 116e of earbud 114a may cause the communications interface 120e of the earbud 114a to emit one or more radio signals to earbud 114b. The processor of earbud 114b may determine the distance to earbud 114a based on the radio signals (and any data included in the signals), e.g., based on one or more of received signal strength (RSS), time of flight (ToF), and angle of arrival (AoA). The earbud 114b may return an indication of the determined distance to earbud 114a via the communications interface 120e. More generally, technique may be used to determine distances between the earbuds 114a and 114b in a given earbud pair 102. Once determined, the distances between the earbuds 114a, 114b (as well as between two or more microphone arrays 208, as these distances are fixed) can be used as points in space to determine the position a sound originated, e.g., using triangulation, trilateration, beamforming, single or multiple microphone acoustic impedance measurements, or impulse/frequency response function measurements, etc.

Because the microphone arrays 208 are placed at known locations, the therapy application 216 and/or the models 218 may use these (or other) algorithms to calculate the exact position of the source of the sound. Doing so may allow the therapy application 216 to detect therapy devices being worn or otherwise used by the patient, detect pathologies, etc. As described herein, the distances between the earbuds 114a, 114b (and the corresponding microphone arrays 208) may be periodically determined, e.g., to facilitate the detection of a pathology using both earbuds 114a, 114b, detection of a device such as RPT device 106, mask 108, or other therapy devices 122 used by the patient, etc. Furthermore, using the models 218, the therapy application 216 and/or the models 218 may adjust the position determination algorithm to compensate for how sounds travel through the airway, how sounds travel through the ear canal, how sounds travel through fluid, how sounds travel through tissue, etc. Doing so allows the therapy application 216 and/or the models 218 to accurately determine the location where a sound originated.

In some embodiments, the therapy application 216 may detect the location of an airway obstruction in the patient based on sounds generated as the airway closes. In some embodiments, the therapy application 216 may determine a degree of the obstruction, e.g., partial, complete, etc. In some embodiments, the therapy application 216 may determine a type of the obstruction based on the sounds, e.g., OSA, positional sleep apnea, etc. In some embodiments, positional sleep apnea is determined based on position information received from the accelerometer 212 and/or one or more other wearables 110, e.g., to determine whether the patient is sleeping on their back, etc.

In some embodiments the therapy application 216 and/or the models 218 may determine whether the determined location is within the body of the patient, e.g., to identify external sounds. For example, the therapy application 216 and/or the models 218 may determine whether at least a portion of the sound was captured by the microphone arrays 208 through the body, e.g., through the airways, through the ear canal, through the tissues of the body, etc. If the therapy application 216 and/or the models 218 determine the sound was captured by the microphone arrays 208 through the body, the therapy application 216 and/or the models 218 may determine that the sound originated from within the body. In such an example, the therapy application 216 /or the models 218 may exclude external therapy devices such as RPT device 106 as being the source of the sound and instead consider internal therapy devices as the source of the sound. Similarly, if the therapy application 216 and/or the models 218 may determine that the sound originated external to the body, the therapy application 216 may exclude internal therapy devices as being the source of the sound, and instead consider external devices such as RPT device 106 as the source of the sound.

In addition and/or alternatively, the therapy application 216 and/or the models 218 may consider the distance to the determined sound location relative to one or more of the earbuds 114a, 114b. For example, if the distance between the determined sound location and one or more of the earbuds 114a, 114b is 10 meters, the therapy application 216 and/or the models 218 may determine that the sound did not originate from within the body, as this distance is too great to originate from within the body. In addition and/or alternatively, the therapy application 216 and/or the models 218 may consider the direction of the sound captured by the microphone arrays 208. For example, if the direction of the sound indicates the sound was generated above and behind the earbuds 114a, 114b, the therapy application 216 and/or the models 218 may determine that the sound did not originate from within the body. The accelerometer 212 may provide position information to facilitate the direction from which the sound originated relative to the body, e.g., the head and/or ears of the body. Doing so may allow the therapy application 216 to determine specific therapy devices worn or otherwise used by the patient. For example, based at least in part on a determination that a sound originated near the patient's face, the therapy application 216 may determine the sound is associated with a mask 108 being worn by the patient. As another example, based at least in part on a determination that the sound originated a meter away from the patient, the therapy application 216 may determine the sound is associated with an RPT device 106 used to deliver therapy to the patient. Embodiments are not limited in these contexts.

Furthermore, the analysis of the sounds may be used to compute an anti-phase sound to cancel the sound, e.g., using active noise cancellation. In one example, the anti-phase sound is computed as a wave with the same frequency and amplitude characteristics of the sound, but with an inverted phase (e.g., 180-degree phase shift). The anti-phase sound may be computed by one or more of the microphone arrays 208, the processor 116e, the therapy application 216, and/or the models 218.

More generally, the therapy application 216 and/or the models 218 may analyze one or more of the sounds detected by the plurality of microphone arrays 208. For example, the analysis may be used to determine a therapy device worn by the patient, determine a pathology associated with the sounds and/or device, determine errors or other problems with therapy devices, etc. For example, the therapy application 216 and/or the models 218 may perform waveform analysis on the soundwaves detected by the microphone arrays 208. For example, the therapy application 216 may compare the soundwaves (e.g., the waveforms, attributes of the soundwaves, etc. ,) to known examples of types of sounds, e.g., sounds associated with devices, sounds associated with pathologies, etc. In some embodiments, the known types of sounds and associated pathologies (and/or associated devices) may be stored in the therapy application 216, e.g., in the device profiles 224. Therefore, if a sound is similar to a stored sound associated with an RPT device 106, the therapy application 216 and/or the models 218 may determine the patient is using an RPT device 106, which may be associated with OSA. As such, the therapy application 216 and/or models 218 may determine the patient has OSA. As another example, if a sound is similar to a stored sound associated with a therapy device such as RPT device 106, mask 108, or other therapy device 122, the therapy application 216 and/or models 218 may determine that the patient is wearing or otherwise using the therapy device.

As stated, in some embodiments, the therapy application 216 determines a device, a pathology, a location of the pathology, and/or any attribute thereof based at least in part on the models 218. The models 218 represent any type of model, such as a machine learning (ML) model, neural network, large language model (LLM), or any other type of artificial intelligence (AI) model. For example, the models 218 may include models trained to identify locations of input sounds, models trained to identify sounds based on input sounds (e.g., sounds of obstructions at a plurality of points in the airway, sounds of types of obstructions, sounds associated with therapy devices, sounds generated by other parts of the human body, etc.), models of the airway, models of the ear canal, models trained to determine how sounds travel through the airway, models trained to determine how sounds travel through the ear canal, models trained to determine how sound travels through tissues, models trained to determine how sounds travel through fluid, and models trained to generate treatments or other recommendations for identified devices, pathologies, etc.

In artificial intelligence embodiments, the models 218 may be trained based on training data, e.g., data describing different sounds (and/or soundwaves), data from a plurality of users, data describing therapies for pathologies, etc. For example, the training data may include sounds (and/or soundwaves), attributes of the sounds and/or soundwaves, etc. The models 218 may be trained to identify features of the sounds such that once trained, the models 218 may return a sound similar to an input sound. For example, the models 218 may be trained to identify the sounds of tissues collapsing in the airway. Based on an input sound of tissues collapsing in the airway, the models 218 may determine that the input sound is similar to the sound of tissues collapsing in the airway. Similarly, the models 218 may be trained to identify other data associated with input sounds, such as associated pathologies, therapy devices, treatments, etc. Therefore, the models 218 may return a device associated with the input sound, a pathology associated with the device, a therapy associated with the device and/or pathology, errors with a device, etc. The models 218 may be retrained over time, e.g., to be tailored to a particular user, device, and/or pathology. Embodiments are not limited in these contexts.

Advantageously, the therapy application 216 may identify devices being worn or otherwise used by a patient based on sounds detected by the microphone arrays 208. For example, by detecting a sound associated with an RPT device 106, the therapy application 216 may determine the type of RPT device 106, an associated pathology, etc. In some embodiments, the therapy application 216 detects the use of a therapy device based on how sounds change over time. For example, an RPT device 106 may cause improvement of airflow in the patient's airway over time. Therefore, the therapy application 216 may detect sounds indicating the patient's airway is opening (and airflow is increasing), which may allow the therapy application 216 to determine the patient is wearing or otherwise using a therapy device. As another example, the sounds generated by a device may change during the course of operation. For example, the RPT device 106 may get louder (or emit higher frequency sounds) while in operation. Therefore, the therapy application 216 may monitor changes in the sounds emitted by a device such as RPT device 106 to determine the patient is wearing or otherwise using a therapy device. In some embodiments, other data such as the oxygen saturation of the bloodstream is used in combination with the detected sounds to determine the patient is wearing or otherwise using a therapy device.

Similarly, the therapy application 216 may determine errors or other problems with the device or component thereof. For example, the therapy application 216 may detect sounds associated with an airflow leak. By detecting the airflow leak and the type of therapy device, the therapy applications 216 may generate one or more recommendations, e.g., to prescribe a new type of therapy device (e.g., a different RPT device 106 such as CPAP or bilevel, a different mask 108 (e.g., a full-face mask 108, oronasal flexible mask 108), different components of RPT devices 106 and/or masks 108), using different cushion types, ordering replacement parts (e.g., cushion types, tubes, etc.), adjust operating parameters of the therapy device (e.g., modifying the coefficients used in quadratic relationships between pressure drops and air flow in the RPT device 106, etc.), etc. More generally, the adjustment of operating parameters of the RPT device 106 may include modifying pressure, changing pressure mode (e.g., fixed pressure mode and/or auto-adjusting (APAP) pressure mode), ramp time, pressure titration, humidification, tidal volume, respiratory rate, inspiratory time, rise time, etc. In some embodiments, the recommendations may be associated with different times and/or conditions. As another example, by detecting an airflow leak or other error in a therapy device, the therapy application 216 may present specific instructions to the user. For example, if the therapy application 216 determines the patient is not wearing a specific type of mask 108 correctly, the therapy application 216 may transmit an indication of the type of the mask 108 to an application on the user's smartphone as part of a request to display instructions (e.g., a specific set of pages of a user manual) on how to properly wear the mask 108 (e.g., how to adjust the straps on the specific type of mask, etc.). Doing so allows the user to selectively view the relevant instructions without having to manually search and find the specific instructions for the specific type of mask 108, e.g., where a support application has instructions or manuals for multiple types of masks 108.

Furthermore, the therapy application 216 may determine the impact a detected sound has on a patient. For example, if the therapy application 216 determines an airflow leak wakes a patient, the correction of the leak may improve the patient's sleep, and therefore the patient's overall health.

More generally, the therapy application 216 may identify devices, pathologies, and/or attributes thereof based on sounds detected by the microphone arrays 208. For example, the models 218 may receive indications (e.g., waveforms, attributes of the waveforms, etc.) of one or more sounds recorded by the microphone arrays 208 as input. The models 218 may process the sounds to determine a location the sound originated from, e.g., using triangulation, trilateration, beamforming, single or multiple microphone acoustic impedance measurements, or impulse/frequency response function measurements, etc. In addition and/or alternatively, the models 218 may determine one or more sounds similar to the input sound (e.g., based on the waveforms and attributes thereof, such as pressure, amplitude, wavelength, frequency, etc.) and a pathology associated with the input similar sounds. For example, based on one or more soundwaves recorded by the microphone arrays 208 (and/or attributes thereof, e.g., pressure, wavelength, frequency, amplitude, etc. ,) of an airway collapsing, the models 218 may determine the sound is associated with an airway collapse. Doing so allows the therapy application 216 and/or the models 218 to identify a pathology at a particular location in the body. For example, the therapy application 216 and/or models 218 may use the location (e.g., at the soft palate), the determined type of sound (e.g., airway collapse), and any other input to determine the pathology is an OSA event.

The models 218 may further determine the location of a sound, a device associated with the sound, and/or any attributes thereof based on one or more of soundwaves (or portions thereof) detected via the ear canal, soundwaves (or portions thereof) detected via the airway, soundwaves (or portions thereof) detected through fluid in the airway and/or ear canal, soundwaves (or portions thereof) detected through human tissue, etc. The therapy application 216 may further use the models 218 to return a recommended therapy and/or treatment for the determined pathology. For example, based on an input pathology (e.g., OSA), the models 218 may return a recommended therapy and/or treatment.

As stated, the therapy application 216 and/or models 218 may consider other information to detect a device, pathology, and/or a location thereof. For example, because the earbuds 114a, 114b are worn in the ear and the accelerometer 212 can provide orientation information, the therapy application 216 and/or models 218 are able to distinguish sounds coming from the airway, from within the ear, external to the body, etc. Therefore, the therapy application 216 and/or models 218 are able to filter out sounds originating from outside the body, etc. Furthermore, the therapy application 216 and/or models 218 may filter or otherwise ignore signals originating from within the body, but are not associated with therapy devices and/or pathologies. For example, some digestion-related sounds may be identified and/or filtered (e.g., as not being associated with a device and/or a pathology).

In some embodiments, the earbuds 114a, 114b may analyze the geometry of the ear canal. For example, the models 218 and/or user profiles 222 may include one or more models of geometry the human ear canal. The models of the geometry of the human ear canal may include models under various conditions, e.g., complete airway obstructions, partial airway obstructions, snoring, coughing, the presence of fluid in the ear canal, using a device (e.g., RPT device 106, mask 108, other therapy devices 122), using a device according to different configurations (e.g., using a mandibular advancement device in a number of different positions), etc. The models of the geometry of the ear canal may include models specific to a patient, and generic models that are not specific to any patients.

To determine a device and/or pathology based on the geometry of the ear canal, the earbuds 114a, 114b may emit one or more vibrations and/or one or more sounds into the ear canal for acoustic reflectometry analysis. As these sounds and/or vibrations travel through the ear canal, some of the waves are reflected back towards the source (e.g., the earbuds 114a, 114b). The microphone arrays 208 and/or haptic feedback modules 210 may detect the reflected waves. The therapy application 216 may use acoustic reflectometry to create a model of the ear canal by analyzing the reflections of the emitted waves to map the geometry of the ear canal, thereby creating a model of the geometry of the ear canal. For example, the timing and intensity of the reflections provide information about the ear canal's shape and size. For example, the therapy application 216 may determine, based on the reflections, the acoustic impedance at different points along the ear canal. The acoustic impedance may be influenced by changes in the cross-sectional area and the presence of any blockages or abnormalities. The therapy application 216 and/or the models 218 may then use an algorithm to create the model of the ear canal. The generated model may then be used to detect differences, e.g., based on models of ear canal geometry stored by the therapy application 216, based on models of ear canal geometry stored by the models 218, previous models of the patient's ear canal generated and stored by the therapy application 216, etc.

For example, the therapy application 216 may compare the model of the ear canal created by the therapy application 216 to one or more stored models of the ear canal stored by the therapy application 216, e.g., in the user profiles 222 or the models 218. The therapy application 216 may determine a stored model that is similar to the input model based on the geometries. For example, if the input model is similar to a stored model of the ear canal during a partial airway collapse, the therapy application 216 may determine the patient has a partial airway collapse. As another example, the model created by the therapy application 216 may be provided as input to the models 218, which may return an indication of a pathology. For example, the features of the model of the ear canal may be similar to the features of an ear canal during a complete airway collapse by the models 218. Therefore, the models 218 may return an indication that the patient is experiencing the complete airway collapse.

As another example, the therapy application 216 may compare the model of the ear canal created by the therapy application 216 to one or more stored models of the ear canal stored by the therapy application 216, e.g., in the user profiles 222 and/or models 218. The therapy application 216 may determine a stored model that is similar to the input model based on the geometries. For example, if the input model is similar to a stored model of the patient's ear canal in the user profiles 222 (which may be associated with using an RPT device 106), the therapy application 216 may determine the patient is using an RPT device 106. For example, the features of the model of the ear canal may be similar to the features of a model of an ear canal during use of the RPT device 106 in the models 218 and/or user profiles 222.

In some embodiments, the models of the ear canal in the user profiles 222 may include models associated with the use of various therapy devices. For example, different models of the ear canal in the user profiles 222 may be associated with the patient using a mandibular advancement device in different configurations (e.g., where different configurations, or positions, of the mandibular advancement device cause the mandible and/or tongue to move forward to different degrees, e.g., by attaching rubber bands to one of several different attachment points on the device, changing pairs of top and bottom sets of mandibular devices, etc.). The different models of the ear canal may reflect the degree to which the mandibular advancement device is changing the shape of the ear canal (e.g., as there may be a proportional change in the geometry of the ear canal as the forward movement of the jaw increases). Thereafter, the therapy application 216 may generate a new model of the ear canal, compare the new model to the stored models, and determine that the mandibular advancement device is being used, and the corresponding position the mandibular advancement device is in (e.g., which of the attachment points are being used).

In some embodiments, the geometry of the ear canal may vary based on having illnesses such as the common cold, the flu, or any illness that causes the buildup of fluid in the ear canal. Advantageously, the therapy application 216 may detect the presence of fluid in the ear canal based on one or more generated models of the ear canal. By detecting the presence of fluid in the ear canal, the therapy application 216 may determine an illness (e.g., the common cold, flu, etc.) that may negatively impact breathing and generate a therapy recommendation. For example, by detecting the presence of fluid in the ear canal (and/or the associated illness), the therapy application 216 may recommend a different type of mask 108. For example, the therapy application 216 may recommend a full face mask 108 over a nasal mask 108 to combat breathing difficulties seen with using only the nasal type mask 108 while sick.

In some embodiments, one or more models of a given patient's ear canal may be stored as a baseline model in the user profiles 222. Thereafter, the therapy application 216 may update the model of the ear canal at predetermined time intervals (and/or in response to detection of a sound associated with a device, a sound associated with a pathology, according to the mode of operation, etc.) and store the updated models in the user profiles 222. As the therapy application 216 creates new models of the patient's ear canal, the therapy application 216 may compare the new models to one or more of the stored models in the user profiles 222 to detect a change in the geometry of the patient's ear canal. Doing so allows the therapy application 216 to detect changes or other abnormalities in the ear canal.

For example, the therapy application 216 may detect a device being worn or otherwise used by the patient. While the patient is awake and wearing earbuds 114a-114b, the therapy application 216 may generate a first model of the ear canal and store the first model in the user profiles 222 for the patient. The first model of the ear canal may include features reflecting that the patient is not wearing a therapy device, e.g., based on decreased airflow, etc. Thereafter, (e.g., during sleep) the patient may wear a therapy device such as an RPT device 106, mask 108, hypoglossal stimulation, mandibular advancement device, etc. The therapy application 216 may generate a second model of the ear canal during use of the therapy device and store the second model in the patient's user profile 222. The therapy application 216 may determine the patient is wearing the therapy device based at least in part on the second model. For example, the therapy application 216 may compare the second model to a plurality of different models 218 of the ear canal of the patient in the user profiles 222 (which may include the first model). The therapy application 216 may determine the second model matches or is otherwise similar to a stored model of the patient's ear canal associated with the use of a particular therapy device in the patient's user profile 222, e.g., the RPT device 106, mask 108, etc. In addition and/or alternatively, the therapy application 216 may determine the patient is wearing the therapy device based at least in part on a determination that the second model is different than the first model.

In some embodiments, the therapy application 216 may determine a therapy device is not being used based on detecting specific sounds. For example, if the therapy application 216 detects sounds associated with teeth grinding (bruxism), the therapy application 216 may determine that the patient cannot be wearing an intraoral device, as such a device may prevent the patient from being able to grind their teeth. Similarly, if the therapy application 216 detects sounds associated with tongue contacting the patient's teeth, the therapy application 216 may determine that the patient cannot be wearing an intraoral device, as such a device may prevent the tongue from contacting the teeth.

Some therapy devices create sounds based on air flow, e.g., RPT device 106 and/or masks 108. However, different devices create different sounds. As such, specific types of RPT devices 106 and/or masks 108 may be determined based on the types of sounds detected by the microphone arrays 208.

Advantageously, the therapy application 216 may determine the use of a therapy device by a patient. Regardless of the technique (or combination of techniques) used to detect the use of a therapy device by a patient, the therapy application 216 may perform any number of operations when the therapy application 216 determines the patient is using a therapy device. For example, the therapy application 216 may record the use of the therapy device in the patient's user profile 222, thereby facilitating adherence tracking to prescribed therapy devices. Similarly, other attributes of the patient may be stored in the user profile 222 with the recorded use of the therapy device, e.g., oxygen saturation values, sleep stages, heartrate, etc. Doing so captures data that can encourage the patient to continue adherence (e.g., by displaying improvements in oxygen saturation values while using an RPT device 106 relative to oxygen saturation values while the patient is not using the RPT device 106, etc.).

For improved adherence, in some embodiments, the therapy application 216 may determine, based on the user profile 222 of a patient, a rate of use of a prescribed respiratory therapy device (or devices) is below a threshold. For example, the user profile 222 of the patient may include a prescription to use the RPT device 106 nightly when sleeping to treat OSA. However, the user profile 222 may include entries indicating the patient is using the RPT device 106 twice per week. The entries in the user profile 222 reflecting use of a given device may be manually entered and/or added by the therapy application 216 based on detecting the use as described herein. Therefore, based on the twice per week use being less than the nightly (or 7 times per week) threshold, the therapy application 216 may determine the patient is not adhering to their therapy. As such, the therapy application 216 may generate, a therapy recommendation to use the respiratory therapy device to treat OSA and transmit the therapy recommendation to an external device 104 such as the patient's smartphone.

The patient's adherence to therapy may further be improved by displaying metrics that demonstrate the improvements provided by using a therapy device. For example, average (or mean) oxygen saturation values may be computed during times the patient is using a respiratory therapy device and when the patient is not using the respiratory therapy device, e.g., based on the oxygen saturation values in the user profile 222, some of which may be tagged as being associated with wearing a therapy device, some of which may be tagged as not being associated with wearing a therapy device. For example, the average oxygen saturation value when the patient is using an RPT device 106 may be 99%, while the average oxygen saturation value when the patient is not using an RPT device 106 may be 90%. Therefore, the therapy application 216 may detect the 99% average while on therapy being greater than the 90% average while not on therapy, and output an indication of the improved oxygen saturation to the patient, e.g., on a smartphone of the patient. For example, the indication may include indications of the oxygen saturation values. The indication may further be combined with other indications, e.g., the use metrics, detected OSA events, etc.

In some embodiments, the therapy application 216 may generate one or more recommendations based on a detected therapy device. For example, the therapy application 216 may determine that the patient is using a respiratory therapy device, and identify the particular device (e.g., based on the sounds captured by the microphones, the patient's user profile 222, the device profiles 224, etc.). In some embodiments, the therapy application 216 may determine errors or other problems with the detected therapy device. For example, the therapy application 216 may determine that one or more detected sounds are associated with errors or other problems, e.g., by detecting a sound that matches a stored sound of an air leak, a restricted tube, etc., in the device profiles 224. Therefore, in such embodiments, the therapy application 216 may determine a corrective action. In some embodiments, the corrective action is stored in or otherwise determined based on the device profiles 224. For example, the corrective action may be any type of action, such as a notification sent to the patient's medical provider (e.g., transmit an indication to prescribe a new type of RPT device 106, mask 108, or any component thereof), a notification sent to the user's smartphone (e.g., to order a new mask 108, to restock replacement parts for the device), displaying one or more pages of the instruction manual for the device on the user's smartphone to assist the user to properly wear the devices, generating an audible alert via the speakers 206 to wake the patient, generating haptic feedback via the haptic feedback module 210 to wake the patient, and/or modifying attributes of the therapy device. For example, the therapy application 216 may transmit instructions to the RPT device 106 that cause the RPT device 106 to adjust titration and/or tidal volume, modify the coefficients used in quadratic relationships between pressure drops and air flow in the RPT device 106, etc. More generally, any number and types of corrective actions may be initiated and/or performed.

Continuing with the example of modifying the coefficients used in quadratic relationships between pressure drops and air flow in the RPT device 106, in some embodiments, an RPT device 106 may include a lookup table that stores the coefficients for the quadratic relationships between pressure drops and air flow. Copies of the lookup tables may be stored in the device profiles 224. A given entry in the lookup table may be associated with a specific type of mask 108 and/or a particular tube of the mask 108. By monitoring sounds associated with the airflow in the tubes of the mask, the therapy application 216 may be able to modify the coefficients and/or select a different entry in the lookup table to improve the overall respiration of the patient. As another example, the therapy application 216 may obviate the need to implement the lookup table. For example, the therapy application 216 may learn the coefficients and instruct the RPT device 106 to implement the learned coefficients.

Stated differently, as the patient breathes in and out, the therapy application 216 may determine the pressure drop between the RPT device 106 and the mask 108, e.g., based on detected airflow sounds, receiving pressure values from the RPT device 106 and/or mask 108, etc. Over time, the mask 108 pressure may change. The pressure of the mask 108 may be the pressure that drives the vent flow. While the vent flow may increase or decrease over time with suboptimal coefficients, constant vent flow (e.g., a constant mass pressure, which may be stored in the device profiles 224) may be detected by changing the coefficients. By identifying the coefficients that cause the vent flow noise to be minimized and/or constant, the therapy application 216 may identify constant mass pressure (e.g., by identifying fluctuation in noise from the vent flow of the mask 108 that is less than a threshold). The therapy application 216 may then cause the RPT device 106 to operate according to the coefficients that cause the constant vent flow, which may deliver better therapy to the patient than using the lookup tables.

In some embodiments, the therapy application 216 may detect a device being used by a patient based on detecting a change in the patient's ear canal based on two or more models of the ear canal, based on the models 218 classifying or otherwise determining the model is associated with a device, etc. Once a device is detected, the therapy application 216 may generate a recommendation. For example, the therapy application 216 may determine a corresponding therapy 220 associated with the device (and/or an associated pathology), and/or transmit an indication of the device and/or pathology via the network 112 as described herein. Embodiments are not limited in these contexts.

Similarly, the therapy application 216 may detect a pathology based on detecting a change in the patient's ear canal based on two or more models of the ear canal, based on the models 218 classifying or otherwise determining the model is associated with a pathology, etc. Once a pathology is detected, the therapy application 216 may determine a corresponding therapy 220 and/or transmit an indication of the pathology via the network 112 as described herein. Embodiments are not limited in these contexts.

Furthermore, in some embodiments, when a pathology and/or associated location thereof is detected, the therapy application 216 may identify an associated therapy. In some embodiments, the therapy application 216 references the therapies 220, which includes associations between one or more pathologies and one or more therapies or treatments. In some embodiments, the models 218 may generate a recommended therapy or treatment based on one or more of the data collected by the earbuds 114a-114b, the pathology identified by the therapy application 216, any attributes of the pathology identified by the therapy application 216, devices being used by the patient and detected by the therapy application 216, attributes of detected devices, and/or the therapies 220.

In some embodiments, the therapy application 216 may use sound detection to detect other pathologies. For example, central sleep apnea may relate to disrupted breathing during sleep due to the brain's failure to send appropriate signals to the muscles that control breathing. Therefore, central sleep apnea may not be accompanied by an obstruction event in the airway. However, the therapy application 216 may detect central sleep apnea based on detecting sounds from the heart (e.g., heartbeats, heart valve sounds, etc.) in the absence of sounds associated with respiration and in the absence of detecting sounds associated with an obstruction event. Whereas obstructive hypopnea may be associated with turbulent noise as the airways narrow, and the flow velocities increase, central hypopnea or apnea may be associated with reduced turbulent, random sounding flow related noise.

In some embodiments, the therapy application 216 may use sound detection to detect hearing loss. Otoacoustic emissions (OAEs) are sounds generated in the inner ear that can be used to assess the health of the ear and screen for hearing loss. OAEs are sounds produced in the cochlea when stimulated by sound. The sound stimulates hair cells in the cochlea, which vibrate and produce a sound that echoes back into the middle ear. Therefore, the therapy application 216 may test the health of the ear by emitting sounds into the ear and detecting the sounds produced by the cochlea and/or hair cells. If sounds are detected, attributes of the sounds (e.g., pressure, amplitude, wavelength, frequency) may be stored in the user profile 222. Doing so allows the therapy application 216 to monitor the attributes over time. For example, if the attributes of the sounds indicate the OAEs are decreasing in strength, the therapy application 216 may detect a degree of hearing loss. If no sounds are detected, the therapy application 216 may determine the patient has hearing loss, a hearing obstruction, etc. In such embodiments, the therapy application 216 may generate a notification and/or recommendation, e.g., to visit a doctor for hearing tests, prescribe hearing aids, etc. Doing so may be useful in detecting hearing loss and prescribing therapy, particularly in young patients such as babies who lack the ability to communicate their inability to hear.

In some embodiments, the therapy application 216 may identify apneas such as OSA as the cause of hearing loss. OSA may cause a reduction in oxygen in the blood and repeated awakenings during sleep. This may injure the hair cells responsible for hearing in the inner ear, resulting in hearing loss. Therefore, by detecting apneas over time and storing indications of the same in the user profiles 222, the therapy application 216 may correlate the apneas with the hearing loss. For example, patterns in the OAEs and apneas may be identified, e.g., as more apneas are detected, the OAEs get softer and/or stop. Similarly, the use of therapy devices may be used to track improvements in hearing. For example, by logging use of therapy devices such as RPT device 106, mask 108, etc., correlations between the use of therapy devices and improved hearing may be determined. For example, if OAEs return or otherwise increase in strength while the patient is using the RPT device 106 and/or mask 108, the therapy application 216 may determine the apneas are causing hearing loss, and the use of the RPT device 106 and/or mask 108 improve the hearing loss. Embodiments are not limited in these contexts.

In some embodiments, the earbuds 114a-114b may be used to predict heart attack and/or stroke based on localized turbulence sounds generated by damaged blood vessels. For example, sounds of turbulent blood flow may be detected by the microphone arrays 208. The therapy application 216 may determine the sounds are similar to the sounds of turbulent blood flow, and determine the patient has high blood pressure and/or damaged blood vessels. By detecting the location of the turbulent blood flow sounds as described herein, the therapy application 216 may predict and/or detect a pathology. For example, if the location of the turbulent blood flow is in the head, the therapy application 216 may predict a cerebrovascular accident (CVA), e.g., a stroke. Similarly, if the location of the turbulent blood flow is within or near the heart, the therapy application 216 may predict a heart attack, other blockage of blood flow, an aneurysm, etc. When the therapy application 216 predicts an adverse event such as a stroke, heart attack, etc., the therapy application 216 may output a notification to the patient, e.g., to wake the patient, to instruct the patient to go to the hospital. The notification may be outputted via the earbuds 114a-114b, the external devices 104 (e.g., a smartphone of the user), the other wearables 110 (e.g., a smartwatch of the user), the RPT devices 106, and/or masks 108. As another example, the therapy application 216 may transmit an indication of the predicted event to the computing device (e.g., one or more of the external devices 104) of the patient's medical provider.

In some embodiments, the therapy application 216 may detect high blood pressure based on the transit time of sounds of the pulse moving from one location (e.g., the heart) to another (e.g., the earbuds 114a-114b). The larger distance between the heart and the earbuds may provide more accurate blood pressure readings, e.g., relative to the distance between the heart and another device such as a smartwatch that is closer to the heart. By detecting high blood pressure, the therapy application 216 may predict stroke, heart attack, etc., as described above.

Similarly, in some embodiments, the therapy application 216 may condition the detection of an airway obstruction based on sounds detected after the sound associated with the obstruction is detected. For example, some obstructions may be complete, meaning no sounds associated with breathing are detected by the microphone arrays 208 (e.g., via the ear canal). Therefore, the therapy application 216 may detect a complete obstruction further based on the absence of sounds associated with breathing coming from the airway. Similarly, partial obstructions may be identified based on the initial sound followed by different sounds (e.g., sounds associated with breathing during a partial obstruction).

When the therapy application 216 detects a device and/or pathology, such as OSA, the therapy application 216 may perform any number and type of operations. For example, the therapy application 216 may cause the speakers 206 to emit noises or music to wake a patient. In other examples, the therapy application 216 may transmit an instruction via the network 112. For example, the therapy application 216 may cause the RPT device 106 to adjust titration and/or tidal volume. As another example, the therapy application 216 may transmit an instruction to the mask 108 to perform an operation (inventors please list some mask-related changes, if any). As another example, the therapy application 216 may transmit an indication of the pathology to the external devices 104, e.g., a smartphone of the patient, a computing device associated with the patient's medical provider, etc. Doing so allows the medical provider to identify the pathology and administer a treatment.

As another example, the therapy application 216 may determine a recommended treatment for the detected pathology. In some embodiments, the therapy application 216 causes the recommended treatment to be administered (e.g., causing RPT device 106 to change titration, causing the RPT device 106 to change tidal volume, causing the RPT device 106 to administer bilevel positive airway pressure therapy, etc.). In some embodiments, the therapy application 216 transmits an indication of the recommended treatment to one or more external devices 104, e.g., to change the type of mask 108 worn by the patient, provide an MRD, etc.

In some embodiments, the earbuds 114a-114b may offload processing to other devices. For example, the microphone arrays 208 may capture sound waves, which may be transmitted with any other data (e.g., distances between earbud 114a, 114b, distances between the microphone arrays 208, etc.) to the external device 104 to determine the use of a therapy device, determine the location of an airway obstruction or detect another pathology, or determine the location a sound originated. Therefore, in some embodiments, the external devices 104 (or other elements of system 100) include instances of the therapy application 216, models 218, and/or therapies 220.

In some embodiments, the therapy application 216 maintains, in the user profiles 222, a counter of airway obstructions or other detected pathologies in a patient. If the counter exceeds a threshold, the therapy application 216 may perform an associated operation, such as recommending a therapy, implementing the therapy, etc.

In some embodiments, the therapy application 216 may receive data from the other devices of system 100. For example, the therapy application 216 may receive an indication from an external device 104 that the patient is being administered a particular therapy or treatment. The therapy application 216 may then monitor the patient for pathologies as described herein. The therapy application 216 may determine whether the particular treatment or therapy is effective, e.g., based on whether the number of detected pathologies increases or decreases while the patient is under the therapy. For example, if the therapy application 216 detects 20 OSA events in a month while the patient is using a first type of therapy and detects 10 OSA events in the next month while the patient is using a second type of therapy, the therapy application 216 may determine the second type of therapy is effective. The therapy application 216 may then transmit an indication of the effectiveness to other devices in the system 100. In some embodiments, the improvements may be determined based on other factors, such as glucagon-like peptide-1 (GLP1) levels detected in the patient, apnea-hypopnea index (AHI) equivalence scores of the patient (e.g., a score reflecting the severity of sleep-disordered breathing), etc.

In some embodiments, the therapy application 216 may further monitor the overall patency of the patient's airway. For example, using sounds detected by the microphone arrays 208, the therapy application 216 may determine the overall degree to which the airway remains open and unobstructed. In some embodiments, the therapy application 216 computes a score for the overall potency of the airway, e.g., based on the number of detected obstructions, etc.

Although some embodiments of the disclosure are discussed with reference to detecting sounds originating within the human body, the disclosure is equally applicable to detecting sounds originating external to the human body. For example, an earbud pair 102 may be used to determine locations of physical objects in the real world. The earbud pair 102 may then output indications of the determined locations, e.g., via the speaker 206, to assist users who are visually impaired or blind. As another example, the technique can be used in military applications, e.g., to display indications of the determined locations of the objects via the external devices 104, output audible indications via the speaker, etc. Further still, the techniques of the disclosure may be used in augmented reality (AR) systems. For example, detected real world objects may be displayed in an AR interface, e.g., on the external devices 104 and/or other wearables 110.

In some embodiments, the earbuds 114a, 114b may operate according to two or more detection modes. For example, a first mode may include the earbuds 114a, 114b monitoring and analyzing sounds as described above. In such an example, a second mode may include the earbuds 114a, 114b periodically emitting sounds into the ear canal via the speaker 206 and/or vibrations into the ear canal via the haptic feedback module 210. The microphone arrays 208 and/or haptic feedback module 210 may detect response signals (e.g., reflections) from these sounds and/or vibrations. The therapy application 216 may then use triangulation, beamforming, trilateration, single or multiple microphone acoustic impedance measurements, impulse/frequency response function measurements, or any suitable location detection algorithm to detect a therapy device, an airway obstruction, pathology, or any attribute thereof. In some embodiments, the therapy application 216 determines a type and severity of an obstruction based at least in part on the vibrations. A third mode of operation may include using the geometry of the human ear canal to detect pathologies. A fourth mode of operation may include using the geometry of the human ear canal to detect therapy devices and/or errors or other associated problems with the devices. A fifth mode of operation may include any combination of the first, second, third, and fourth modes of operation.

In some embodiments, the therapy application 216 may cause the earbuds 114a, 114b to change between the different modes of operation. The therapy application 216 may change the modes of operations according to any number and type of criteria. For example, the therapy application 216 may change the mode of operation at predetermined time intervals. In addition and/or alternatively, the therapy application 216 may change the mode of operation based on attributes of the waveform of a detected sound (e.g., pressure, amplitude, wavelength, frequency, etc.). For example, if the amplitude of a sound detected by the microphone arrays 208 exceeds a threshold amplitude associated with snoring, the therapy application 216 may change the mode of operation of the earbuds 114a, 114b. As another example, the amplitude of a detected sound may be associated with airflow through the tube (or conduit) of a mask 108, and the therapy application 216 may determine the specific type of mask based on the amplitude of the detected sound. As another example, if the therapy application 216 detects a change in the geometry of the ear canal, the therapy application 216 may change the mode of operation to cause the earbuds to listen for sounds associated with pathologies and detect the location of the sounds as described herein. As another example, if the therapy application 216 detects a change in the geometry of the ear canal, the therapy application 216 may change the mode of operation to cause the earbuds to listen for sounds associated with therapy devices and detect the location of the sounds as described herein.

In some embodiments, the external devices 104, RPT devices 106, other wearables 110 and/or masks 108 include instances of the therapy application 216, not pictured in FIG. 1 for the sake of clarity. In such embodiments, the instances of the therapy application 216 on the earbuds 114a-114b communicate with the instances of the therapy application 216 on the external devices 104, RPT devices 106, other wearables 110, and/or masks 108 (e.g., to transmit notifications, recommendations, tasks, goals, or any other type of content). In some embodiments, the therapy applications 216 may transmit notifications via an operating system (not pictured) of the external devices 104, RPT devices 106, other wearables 110 and/or masks 108. Embodiments are not limited in these contexts.

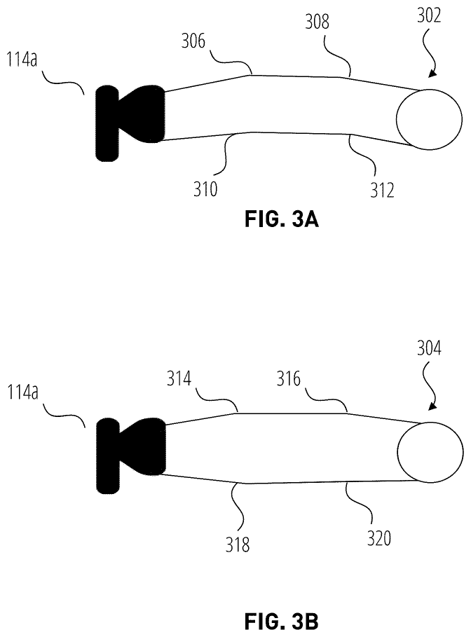

FIG. 3A illustrates a graphical representation of a model 302 of an ear canal generated by the therapy application 216 of an earbud 114a, according to one embodiment. Although the earbud 114a is depicted in FIG. 3A, the earbud 114a is not part of the model 302. As shown, the model 302 may be defined at least in part by a plurality of points (or coordinates) including a point 306, a point 308, a point 310, and a point 312. Although a certain number of points are depicted in FIG. 3A-3B, any number of points may be used to generate the models 302, 304 of an ear canal. The model 302 may therefore be generated based at least in part on the points 306-312. The model 302 and/or points 306-312 may be useful to detect changes in the geometry of the ear canal. The model 302 and/or points 306-312 may be stored in the user profile 222 for the user.

FIG. 3B illustrates a graphical representation of a model 304 of the ear canal generated by the therapy application 216 of an earbud 114a, according to one embodiment. As with model 302, the earbud 114a is not part of the model 304. As shown, the model 304 a point 314, a point 316, a point 318, and a point 320. The model 304 may therefore be generated based at least in part on the points 314-320. The models 302, 304 are of the same ear canal (not pictured for clarity).

The model 304 is generated at a different time than model 302. As such, the therapy application 216 and/or the models 218 may analyze the models 302, 304 to detect a change in the geometry of the ear canal. For example, the change may be based on the total volume of the ear canal, the shape of the ear canal, distances between the two or more of the points 306-320, etc. For example, the distance between points 306 and 310 may be different than the distance between points 314, 318 (where point 306 is proximate to point 314 and point 310 is proximate to point 318). Based at least in part on the different distances, the therapy application 216 may determine a change in the geometry of the ear canal.

In some embodiments, based on detecting the change in the ear canal, the therapy application 216 may determine the patient is wearing or otherwise using a therapy device. In some embodiments, the therapy application 216 may further consider the oxygen saturation values captured by the pulse oximeter 214 when each model 302, 304 was generated to determine the patient is wearing or otherwise using a therapy device. For example, if the oxygen saturation values observed when model 304 was generated are greater than the oxygen saturation values observed when model 302 was generated, the therapy application 216 may determine one or more therapy devices are being used, e.g., to improve respiration and increase the oxygen saturation values.

As stated, in some embodiments, the therapy application 216 may further process sounds detected by the microphone arrays 208 to determine the therapy device is being worn or otherwise used by the patient. For example, the therapy application 216 may determine a sound detected by the microphone arrays 208 is located proximate to the patient's head and matches the stored sound of a therapy device in the device profiles 224. The therapy application 216 may then determine the specific type of therapy device being used by the patient.

More generally, the models of the ear canal, the oxygen saturation values, sound processing, position information, or any combination thereof may be used to detect a therapy device being worn or otherwise being used by the patient. When a therapy device is detected, the therapy application 216 may generate one or more recommendations as described in greater detail herein.

FIG. 4 shows a system including a patient 401 wearing a patient interface 403, in the form of nasal pillows, receiving a supply of air at positive pressure from an RPT device 106. The patient interface 403 represents the mask 108. Air from the RPT device 106 is humidified in a humidifier 405, and passes along an air circuit 404 to the patient 401. A bed partner 402 is also shown. The patient interface 403 is one example of a patient interface, or mask 108. Other examples include, but are not limited to, a nasal mask, a full-face mask, etc. As shown, the patient 401 is wearing earbud 114a (and corresponding earbud 114b, which is not depicted for clarity). In one or more embodiments, the earbuds, patient interface 403, RPT device 106, and humidifier 405 form a respiratory therapy system for treating a respiratory disorder.

As shown, a soundwave 406 may be detected by the microphone arrays 208 of the earbud 114a and/or 114b. The location the soundwave 406 originated may be determined by the therapy application 216 as described herein. Furthermore, the soundwave 406 and/or other soundwaves may be used by the therapy application 216 to generate a model of the ear canal of the patient 401. Further still, the pulse oximeter 214 of the earbud 114a may capture one or more SpO₂ values of the patient.

The therapy application 216 may analyze the model of the ear canal (e.g., by comparing the model to one or more stored models of the patient 401 in the user profiles 222) to detect a change in the geometry of the ear canal (e.g., to determine the model of the ear canal is similar to a model of the ear canal while using the RPT device 106 and/or patient interface 403, to determine the model of the ear canal is associated with improved respiration, etc.). Similarly, the therapy application 216 may compare the SpO₂ values to one or more stored SpO₂ values in the user profile 222 for patient 401, and determine the SpO₂ values are greater than the stored SpO₂ values in the user profile 222 (and/or match stored SpO₂ values associated with the use of RPT device 106 and/or patient interface 403 in the user profile 222). The therapy application 216 may determine the use of the RPT device 106 and/or patient interface 403 by the patient 401 based on the detected change in the geometry of the ear canal, the analysis of the captured SpO₂ values, and the analysis of the soundwave 406.

In some embodiments, the analysis of the soundwave 406 includes determining an error or other problem with the air circuit 404. For example, the soundwave 406 may reflect an air leak in the air circuit 404. As such, the therapy application 216 may generate a recommendation, e.g., to replace the air circuit 404. The type of air circuit 404 may be determined based on the user profile 222 and/or by detecting the type of the RPT device 106 (e.g., based on processing sounds made by RPT device 106) and identifying one or more types of air circuits 404 associated with the RPT device 106. In some embodiments, the therapy application 216 may transmit an instruction to the smartphone of the patient 401 to order the replacement air circuit 404 without requiring user input. More generally, any number and type of recommendation may be generated. As another example, the analysis of the soundwave 406 may reflect a blockage in the air circuit 404 (which may be caused by the sleeping position of the patient 401). In such an embodiment, the therapy application 216 may generate a noise via the speakers 206 of the earbuds to wake the patient 401 such that the blockage is removed. Embodiments are not limited in these contexts.

In some embodiments, a system 504 may be provided for measuring physiological parameters, e.g., characteristics of a patient while they are awake. The system may be configured to provide a patient 503 with ongoing monitoring of their waking physiological parameters, e.g., an “awake state” to determine, e.g., quantify, whether their sleep quality improves as a result of respiratory therapy, e.g., PAP therapy. In this regard, the system 504 may be considered a patient tracker 504 configured to measure a patient's physiological state during the day, rather than at night.

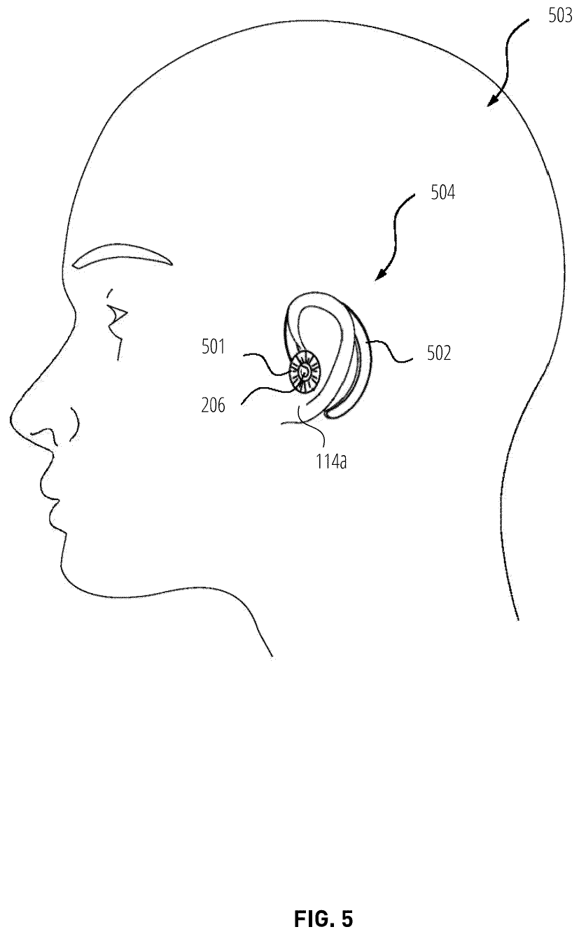

Referring to FIG. 5, the system 504 may be implemented in the form of a wearable device, such as the “earbud” type wearable device shown wearable with respect to a person's ear canal, ear lobe or behind the person's ear. In this regard, the patient tracker 504 may be considered a patient tracking device 504 (also referred to as device 504). Therefore, in at least one embodiment, the device 504 is the earbud 114a and/or earbud 114b.

The patient tracking device 504 may be configured to measure daytime activities and physiological characteristics, e.g., conditions, of the patient. For example, the system may be configured to measure activities such as: a distance travelled by the patient; a number of steps (or paces) walked, run, climbed, etc. ; a type, duration, intensity, etc., of physical activity; time spent standing.

The daytime activities set forth above may influence the physiological characteristics of the patient. For example, a patient travelling a distance may have an elevated heart rate, increased breath rate, etc. The physiological characteristics that can be measured by the system include: a respiration rate, variability of respiration, etc.; a heart rate, variability of heart rate, etc.; a magnitude of calories burned; blood oxygen saturation; electrodermal activity (e.g., skin conductance or galvanic skin response); or any combination thereof.

Referring to FIG. 5, the device 504 may be used by itself, or independently of, e.g., without a the RPT device 106. In particular, the device is shown without a patient interface 601. In this form, the patient may, for example, wear the device by itself during daytime activities such as walking, running, etc.

By comparison, and as shown in FIG. 6, the device 504 may also be used together with an RPT device such as RPT device 106. In the form shown, the device is worn together with a patient interface 601 (as part of the RPT device 106). The patient may wear the device in this way, e.g., with the patient interface 601, while they sleep for recording data while also receiving respiratory therapy.

As shown in FIG. 5 and FIG. 6, the device 504 comprises a body 501 for housing the control system, memory device, sensors, batteries (rechargeable or replaceable), etc. An ear hook 502 is provided for locating, e.g., attaching, mounting, etc., the device 504 about the patient's ear. In particular, the ear hook is configured, e.g., shaped, to locate and removably secure behind the patient's external ear (e.g., auricle/pinna).

The body 501 is configured to locate within the patient's ear for transmitting audio (e.g., sound) into the ear for the patient to hear. At least a portion of the body 501 may be configured to releasably secure within at least a portion of the patient's external auditory canal. In this regard, the body of the device may be shaped similar to a traditional earbud used for transmitting audio into a patient's ear.

The patient tracker 504 may be configured to receive the physiological data about the patient from the one or more sensors (e.g., sensors 204, not pictured for clarity). In some forms, the patient tracker may also be configured to receive environmental data from the one or more sensors.

The environmental data may relate to environmental conditions surrounding the patient (e.g., the environmental data being related to the patient), such as temperature, humidity, etc. In either form of data, e.g., physiological or environmental, the data may be stored in the memory device and analyzed by the processor(s) of the control system.

Advantageously, measuring and recording data relating to the environmental conditions surrounding the patient may allow the device 504 to accommodate for environmental conditions that influence the physiological conditions of the patient. For example, if the humidity and temperature of air surrounding the patient is high, the patient may fatigue more rapidly when e.g., walking, than in colder, less humid conditions. In effect, environmental conditions (such as high humidity and temperature) may inadvertently indicate the patient is fatigued as a result of e.g., a lack of sleep. Hence, allowing the device 504 to accommodate for such environmental conditions means that indications of the patient's sleep quality can be more accurately presented to the patient.

For example, an optical sensor using red, infrared, and/or green, could be used to calculate a photoplethsmogram. Subsequently, parameters such as pulse rate (PR), pulse rate variability (PRV), SpO₂ can be determined. If respective sensors are placed on a periphery of the user, e.g., at their skin, the peripheral arterial tone may also be measured.

Generally, the types of sensors 204 utilized in the patient tracker 504 may vary according to the physiological and/or environmental data being generated. For example, when the patient tracker 504 is integrated into an item of clothing, it may comprise the electromyography (EMG) sensor for detecting electrical signals generated by muscles. Alternatively, the EMG sensor may not be utilized when the patient tracker is integrated into a ring. In any case, each of the one or more sensors may be configured to output sensor data that is received and stored in the memory device of the patient tracker 504.

In some forms of the device 504, one or more of the sensors set forth above may be configured to contact the patient's skin. In this regard, the sensors may be located on an externally facing surface of the body 501 or the ear hook 502, so as to be in contact with the patient's skin when in use. This allows, e.g., the galvanic skin response (GSR) sensor, to measure changes in sweat gland activity on the skin. In another example, the one or more sensors, e.g., optical sensor, may be located on the ear hook so as to contact an area of skin between the patient's auricle/pinna and hairline.

Other forms of the sensors may be configured for mounting internally to the body 501 and ear hook 502, such as the motion sensor. In this case, for example, the motion sensor may be integrated within the body of the device and configured to measure a patient's head movement.

As set forth above, the one or more sensors of the patient tracker 504 can be configured to determine an awake state of the patient. The patient tracker utilizes the physiological and environmental data generated from the sensors to determine how “awake,” e.g., alert, the patient is for a duration of non-sleep, e.g., during the daytime. For example, the device 504 may be configured to measure a patient's heart rate and EEG during the daytime.

Based on the physiological data generated from variations in the heart rate and EEG measurements, the system 504 may indicate how awake the patient is, e.g., if the patient is lethargic and has an unfocussed attention during the daytime.

In order to determine an awake state, including stages of a sleep (such as NREM (N1, N2, N3/SWS) or REM), data may be fed into an artificial Intelligence (AI) or Machine Learning (ML) model such as the one or more of the models 218. This model 218 may be trained on the IMU and PPG signals, or pre-processed parameters of those.

Breathing and/or respiration signal related parameters can include: variability of breathing rate throughout the day and/or night (the variability being characteristic of the person)—this can be inter-breath or over longer timescales—e.g., 30, 60, 90 sec or much longer periods; the stability over time (related to the variability); the standard deviation of breathing rate; the depth of respiration (shallow, deep etc.), and relative amplitude of adjacent breaths; the mean or average value of the breathing rate; the trimmed mean (e.g., at 10%) to reject outliers; wake or Asleep (e.g., the detected sleep stage of the person); surges (sudden accelerations or decelerations) in breathing rate seen during quiet periods and during REM sleep; median (50th percentile); interquartile range (25th-75th percentile); 5th-95th percentile; 10th-90th percentile; shape of histogram; skewness; kurtosis; peak frequency over time; ratio of second and third harmonics of peak frequency; percentage of valid data (Valid Physiologically Plausible Data); autocorrelation of the individual signals; characteristic patterns in the spectrogram; wake or asleep; relative percentage of REM and deep sleep.

Cardiac signals can be processed to produce features such as: heart rate variability HRV (inter beat (e.g., as derived from the Ballistocardiogram) and over longer defined moving windows—e.g., 30, 60, 90 sec); variability over time (interbeat/breath variability); mean; trimmed mean (10%); standard deviation; median (50th percentile); interquartile range (25th-75th percentile); 5th-95th percentile; 10th-90th percentile; shape of histogram; skewness; kurtosis; stability over time; peak frequency over time; ratio of second and third harmonics of peak frequency; percentage of valid data (Valid Physiologically Plausible Data), wake or asleep; autocorrelation of the individual signals; characteristic patterns in the spectrogram.

Cardiorespiratory signals can be formed, such as: magnitude square cross spectral density (in a moving window); cross coherence; respiratory sinus arrhythmia peak; low frequency (LF)/high frequency (HF) ratio to indicate autonomic nervous system parasympathetic/sympathetic balance (LF is often defined as around 0.04-0.15 Hz, whereas HF is around 0.15-0.4 Hz); the cross correlation; cross coherence (or cross spectral density) of the heart and breathing signal estimates; non-linear estimates such as entropy measures; the characteristic movement patterns over longer time scales, e.g., the statistical behavior observed in the signals; patterns of movement during detection of and comparison of these heart and breathing signals (e.g., during sleep, some people may have more restful and some more restless sleep).

Based on the determination of a patient's awake state, the patient tracker 504 may provide the patient with an indication of how effective their respiratory therapy, e.g., PAP therapy, is at improving their sleep quality. For example, in the case where physiological data indicates the patient is lethargic and unfocussed, such an indication may be correlated with a low efficacy of the patient's PAP therapy.

Conversely, in the case where physiological data indicates the patient has improved capacity for daytime activities, e.g., a lower resting heart rate, etc., such an indication may be correlated with a high efficacy of the patient's PAP therapy. As set forth in more detail herein, the patient tracker may be configured to alert the patient of such indications, e.g., notifications that e.g., a morning run, positively impacted their sleep.

The patient tracker 504 may be configured to measure an efficacy of respiratory therapy by recording a baseline measure of “off therapy” physiological data and comparing this to an “on therapy” measure of physiological data. According to the changes detected in the measured data, the patient tracker may advise the patient of either improvements to their sleep performance, or deteriorations to their sleep performance.

In a variation, the patient may be advised of improvements that occur in their ability to undertake daytime activities, such as capacity for exercise, that are a result of their corresponding improvements to their sleep performance. Conversely, the patient tracker can be configured to notify the patient of a deteriorated capacity to perform daytime activities as a result of a corresponding deterioration in their sleep performance. Advantageously, notifying a patient of said changes to either their sleep performance or capacity for daytime activities can allow a patient to understand an impact of their respiratory therapy.

As part of providing the patient with an indication of how effective their respiratory therapy is, the patient tracker 504 may also be configured to record, e.g., “timestamp” events associated with the patient's sleep periods. For example, the various sensors of the patient tracker 504 may be configured to record a time that the patient wakes after a period of sleep, times when the patient wakes during a period of sleep (e.g., a rate of sleep disturbances), a time that the patient exits the bed, a time that the patient enters the bed, etc. These events may be utilized, e.g., analyzed, together with other sensor data gathered about the patient, to determine how awake the patient may be as a result of their e.g., PAP therapy.

Advantageously, data relating to e.g., when a patient wakes, may be longitudinally recorded so as to determine sleeping patterns of the patient. This information may be processed and utilized to inform the patient of e.g., whether they are ready for sleep; whether they are sleeping well; whether they should expect to feel tired during their waking hours, etc. Ultimately, the patient tracker 504 may provide the patient with an indication of how effective their respiratory therapy has been.

Set forth below are some further examples of sensors such as sensors 204 that may be used with the patient device 504, and their application for use with the patient device 504.

In some forms of the patient tracker 504 where the motion sensor is utilized (as set forth previously), the motion sensor may generate data relating to specific movements of the patient, such as exercise (e.g., running), or other body (e.g., limb) movements. These movements may be utilized to determine the patient's awake state. For example, a patient's limb movements may be analyzed and determined as being slow relative to a standard measurement of the patient's normal movements.

While the motion sensor is described in broad terms, the motion sensor may be specifically one or more inertial sensors, such as accelerometers, gyroscopes, and magnetometers. These types of motion sensors may be selected, e.g., utilized according to their optimal use-case.

In some forms of the motion sensors, the motion sensors may be configured to detect motion or acceleration associated with arterial pulses, such as pulses in or around the face of the patient and in particular, those proximal to the patient tracking device 504, e.g., the body 501. The motion sensors in this form may be configured to detect features of the pulse shape, speed, amplitude, or volume that may be analyzed to indicate qualities of a patient's awake state.

In other forms, an EEG sensor may also be provided in the patient tracker 504 for measuring physiological data relating to the patient's brain. The EEG sensor may include one or more dry electrodes positioned on or around the scalp of the patient. In this form, the EEG may locate within, or extend from, a portion of the ear hook 502 or body 501. For this reason, the EEG sensor is optimally utilized when the patient tracker is implemented as an earpiece, as shown in FIG. 5 and FIG. 6, such that the external surfaces of the body 501 and ear hook 502 may be in contact with the patient's scalp.

Depending on the placement of the EEG sensors, it may be possible to detect EEG slowing during the daytime (such as a higher ratio of delta and theta frequencies to alpha and beta frequencies) and relate this daytime slowing to greater daytime sleepiness. Thus, it may be possible to avoid asking a patient if they have daytime sleepiness, but rather, derive it from EEG slowing vs. a baseline and relate this to a reduced movement of the patient (detected from e.g., an accelerometer).

In forms where a PPG sensor is provided to measure, e.g., a heart rate, the patient tracker is optimally configured to contact the patient's skin. In this form, the patient tracker may be integrated into a piece of clothing to optimally generate data relating to e.g., a heart rate pattern, a heart rate variability, a cardiac cycle, respiration rate, estimated blood pressure, or any combination thereof.

When the patient tracker is integrated into an earbud and/or earpiece as shown in FIG. 5 and FIG. 6, a speaker 206 may be provided for outputting (e.g., generating) audio. The audio, e.g., generated sounds, are configured to be projected into the patient's ear so as to be heard by the patient. For example, the patient tracker 504 may be configured as a type of earphone to play music for a patient to listen to during the day. In another example, the patient tracker 504 may be configured to sound an alarm for waking the patient from sleep, reminding them of an event (e.g., a calendar event). In yet a further example, the device 504 may assist in relaxation of the patient prior to sleep by playing controlled breathing audio cues. In yet a further example again, the device 504 may also provide hearing assistance, whereby the device may be coupled with a smartphone to generate audio, amplify audio, etc.

In some implementations, the speaker 206 may be used together with, or substituted by, a bone conduction speaker. In this form, the bone conduction speaker is not configured to generate audio for the patient to hear via their ears, rather, the speaker generates vibrations that are configured to penetrate the patient's temporal bones. In variations, an audio and bone conduction speaker may be configured for use together.

In some further implementations, the speaker 206 may be a noise cancelling speaker for assisting in reduction of background noises. Advantageously, this may be used prior to sleep, for reducing background noises that may otherwise hinder sleep.

In either form of the speaker, e.g., as a speaker, bone conduction speaker, noise cancelling speaker, etc., the patient tracking device 504 may be coupled (e.g., wired or wirelessly) to a computing device such as external device 104, e.g., a mobile phone, for playing music or otherwise generating the sounds for the patient to hear. In the case of a wireless connection, the patient tracking device 504 may be configured to communicate through various communication protocols, such as, Wi-Fi, Bluetooth, etc. The patient tracking device may thereby include an antenna, a receiver, a transmitter, a transceiver, or any combination thereof for communicating with wirelessly with a computing device.

The external device 104 may be configured to operate, e.g., run software configured to communicate with the patient tracking device 504. In forms where the external device 104 is a mobile phone or tablet, the software may be configured as a mobile application allowing the patient to control operation of the patient tracking device via the mobile device.

The external device 104 may be used as a way to display information about the patient's awake state or other data in the user profiles 222. In other forms, the computing device may also be configured to process (via one or more processors) data generated from the patient tracking device 504. In further forms, the external device 104 may be configured to receive input from the patient for controlling operation of the patient tracking device. As set forth above, the input of the patient may relate to the patient configuring the patient tracking device 504 to send diary alarms, or in other cases, to select music to listen to (via the speakers).

In some forms of the patient tracking device, the patient may input information into the computing device, e.g., via the software, for determining, at least in part, the awake state of the patient. That is, the patient may self-report information that may not be sensed, per se, but be provided by the patient to be considered together with physiological and/or environmental data generated by the sensors. The combination of self-reported data and sensed data may be analyzed to determine a patient's awake state.

The self-reported information input by the patient may include demographic information, biometric information, therapy device use, medical information such as medications, etc., diet(s), subjective stress level of the patient, subjective fatigue level of the patient, subjective health status of the patient, a recent life event experienced by the patient, or any combination thereof. In the case of the medical information, the patient may provide information relating to one or more medical conditions, medication usage, etc.

Referring to FIG. 6, the patient tracking device 504 (e.g., earbud 114a or 114b) may be configured for use with respiratory therapy, e.g., an RPT device such as RPT device 106. The RPT device may include the patient interface 601, a conduit 603, a mask 108 and a positioning and stabilizing structure 602. It is noted that although a particular mask is shown in FIG. 6, other types of masks may be utilized, such as a full-face mask, nasal mask, oro-nasal mask, etc.

As set forth above, the patient tracker 504 may provide the patient with ongoing monitoring of their “awake” state and provide feedback to the patient regarding any differences detected between “on” and “off” therapy. In other words, the patient tracker 504 may indicate changes in the patient's sleep performance after they begin respiratory therapy and, in effect, indicate to the patient how effective their use of respiratory therapy has been.

The patient tracker 504 may be configured to correlate changes in a patient's daytime activities with their adherence and/or compliance to e.g., CPAP therapy. For example, in patients having symptoms such as chronic fatigue, daytime sleepiness, cognitive impairment, etc., the patient tracker (as set forth previously) may be configured to monitor for improvements in such symptoms. The patient tracker may be configured to determine correlations between these improvements, e.g., changes, and the patient's adherence and/or compliance to CPAP therapy. These correlations may be reported, e.g., communicated as feedback to the patient, so that the patient is aware of the positive impact their adherence and/or compliance to CPAP therapy has on their capacity for performing daytime activities.

The patient tracker 504 may be configured to interrelate a specific respiratory therapy, e.g., CPAP and a deterioration of healthy behaviors or an improvement of healthy behaviors. That is, the patient tracker 504 may also be configured to monitor and report to a patient their unhealthy behaviors which may occur as a result of sleep related breathing disorders.

For example, patients having sleep apnea for an extended period of time prior to diagnosis may develop unhealthy behaviors, such as lack of exercise, bad sleep habits, etc., which may persist even after commencing respiratory therapies, e.g., CPAP. The sensor(s) and self-reported information input into the patient tracker 504 may be used to monitor and report to the patient such behaviors. Reporting these behaviors as feedback to the patient may assist the patient to change, e.g., re-train such behaviors. Advantageously, re-training the patient to remove said unhealthy behaviors can positively impact their respiratory therapy, in addition to reducing the patient's risk of comorbidities.

In this regard, the patient tracker 504 can provide the patient with a complete treatment for their sleep related breathing disorder(s). That is, in addition to opening the patient's airways via, e.g., PAP therapy, the patient tracker can identify and treat unhealthy behaviors that are symptomatic of the sleep related breathing disorder. Advantageously, this can motivate a patient to be more adherent and/or compliant to respiratory therapy.

In some forms, the patient tracker 504 may be configured to provide the patient with detailed correlations of their improved daytime activities and corresponding compliance to respiratory therapy. For example, the patient tracker 504 may be configured to record use of therapy devices and correlate a patient's use of CPAP therapy during a sleep period, with the patient being able to run a larger distance the following day, or the patient having a lower resting heart rate, etc.

The patient tracker 504, as set forth above, can be configured to improve a patient's adherence and/or compliance by behavioral intervention. That is, the patient tracker 504 can be configured to allow a patient to break, e.g., intervene, particular habits that are associated with their sleep related breathing disorder(s).

In some forms, a patient's compliance and/or adherence to a respiratory therapy may also be detected by measurements taken by the one or more sensors of the patient tracker 504. For example, the patient tracker may include an EEG configured to measure daytime markers of a patient's increased alertness. Such markers may be compared against normal measures of the patients'alertness, such that an indication of the patients'improved alertness can be determined. This can indicate an improved efficacy of the respiratory therapy, and in turn, indicate the patients'adherence and/or compliance to therapy. Advantageously, use of the sensors to automatically detect efficacy and therapy adherence and/or compliance means that the patient may not be required to monitor their perceived daytime sleepiness, e.g., lethargy or reduced alertness to determine an efficacy of their respiratory therapy.

Furthermore, utilizing the EEG for compliance indications may also allow for a detection of impaired cognitive function. That is, detection of a patient's alertness may be used as a proxy for an assessment of their cognitive function.

In some forms, the patient tracking device 504 may be coupled with the RPT device 106 to monitor the patient's sleep stage during periods of sleep. In this form, the sensors of the patient tracking device 504 may be used together with the sensors of the RPT device 106 (e.g., optionally located in the patient interface 601, flow generator, or other component of the RPT device 106), for detecting e.g., states of a sleep cycle. In some forms, the data collected may be used to inform the patient of how effective their respiratory therapy has been, and in other forms the data may be additionally or alternatively used to adjust the delivery of respiratory therapy, e.g., pressure, flow rate, etc. Therefore, the earbuds 114a, 114b, and/or the therapy application 216, may instruct the RPT device 106 to adjust pressure, flow rate, etc., based on a pathology detected by the therapy application 216.

As stated, one or more microphone arrays 208 may also be provided to the patient tracking device 504 to measure a patient's breathing during sleep. In this form, a microphone array 208 may be located proximal to the patient's mouth and/or nose, and so accurately record breathing sounds. A detection of abnormal breathing may be indicative of a sleep apnea, whereby the patient tracking device (e.g., earbuds 114a-114b) may be used together with the RPT device 106 to adjust therapy, e.g., pressure, flow, etc., for stimulating a change in the patient's breathing.

In some further forms, one or more motion sensors 204 described previously may be utilized during a patient's sleeping period to detect movements of the patient. For example, a number of movements during a sleep period may be detected, and used to provide an indication of e.g., a disturbed sleep. In some forms, the data collected from the motion sensor may be fused, e.g., coupled, combined, integrated, etc., with flow data collected from the RPT device 106. This combination of data may be used to improve sleep and/or wake classification, e.g., determination of a patient's awake state.

In other forms, the patient tracker 504 may be configured to monitor the patient's sleep stage without being coupled to the RPT device 106. In this form, the patient tracker 504 may be configured to detect and record physiological and/or environmental data as it would when coupled with the RPT device 106. However, rather than adjust operation of the RPT device, the patient tracker 504 in this form would utilize the data recorded to inform the patient of their sleep performance, e.g., apnea events, etc.

In some further forms where the patient tracking device 504 is used without being coupled to the RPT device 106, the data collected during a sleeping period may be implemented as a change to respiratory therapy at a later date. That is, the data collected when the patient is not wearing the patient interface 601 may be used to adjust therapy the next time the patient wears the patient interface 601.

In some further forms, the patient tracking device 504 may be configured to intermittently couple with the RPT device 106 so as to communicate with the RPT device. The patient tracking device 504 in this form may be configured to operate both together with the RPT device 106, and independently of the RPT device 106. That is, when the patient is near the RPT device 106, the patient tracker 504 may be able to connect (e.g., wirelessly) with the RPT device. When the patient is away from the RPT device 106, e.g., walking outside, the patient tracker may be able to operate independently of the RPT device 106.

For example, the patient tracker 504 operating independently may be able to temporarily record and store data from the sensors for later communicating said data to the RPT device 106 when the patient tracker 504 is proximal to the RPT device 106.

In this form, the patient tracking device 504 may be worn together with the patient interface 601 in some instances, e.g., during sleep, and in other instances the patient tracking device may not be worn with the patient interface 601, e.g., when a patient leaves their home. In some cases, missing data e.g., data which is not collected by either the RPT device 106 or patient tracking device 504, may be collected from an alternative data source, such as a wrist worn accelerometer or HR sensor. For example, the device 504 may be coupled with an external device 104 such as a smart watch, or a smart hub for collecting data that may not be captured by the device 504 or the RPT device 106.

In some forms, the external device 104, e.g., mobile device, as set forth previously may be configured to connect with the patient tracker 504 when the patient tracker 504 is not coupled with the RPT device 106. In this regard, the device 504 may be configured to log and process data within its memory, without requiring a wireless connection for a period of time.

In some forms, the patient tracker 504 may be utilized for detecting and diagnosing a patient with an un-treated sleep related breathing disorder. The patient tracker 504 used in this form may allow a patient to determine whether they require respiratory therapy e.g., PAP therapy, positional therapy, insomnia treatment, etc. In this form, the sensors (as set forth previously) may be configured to register (e.g., detect) a sleep event that is indicative of a sleep related breathing disorder.

In forms whereby the patient tracker 504 is configured for detecting and diagnosing a patient with a sleep related breathing disorder, the patient tracker 504 may be utilized to monitor a patient's daytime activities to determine indications of sleep related breathing disorders. For example, a patient may develop unhealthy behaviors, such as lack of exercise, bad sleep habits, etc., that may be detected and utilized as an indicator of insomnia, etc.

In some embodiments, the patient tracker 504 may be utilized for detecting a patient with an under-treated sleep related breathing disorder. That is, a patient having already been diagnosed with a sleep related breathing disorder, but is not receiving effective therapy. In this case, the patient tracker 504 may be configured to monitor e.g., heart rate variability for indicating whether the patient is under-treated. In response, the patient tracker 504 may be configured to provide the patient with an indication of how to adjust therapy during the night, or alternatively, the patient tracker may be configured to automatically adjust a respiratory therapy device (as set forth previously) to appropriately treat the under-treated disorder.

In some forms, the patient tracker 504 can also be configured for detecting and monitoring for comorbidities of sleep apnea. For example, the sensor(s) set forth above may be configured for detecting diabetes, heart failure, stroke, and obesity.

As stated, the earbud pair 102 may be used to detect the use of therapy devices such as RPT device 106, mask 108, conduit 603, etc. As shown, a soundwave 604 may be detected by the microphone arrays 208 of earbud 114a (and/or earbud 114b, not pictured). The location the soundwave 604 originated may be determined by the therapy application 216 as described herein. Furthermore, the soundwave 604 and/or other soundwaves may be used by the therapy application 216 to generate a model of the ear canal of the patient 503. Further still, the pulse oximeter 214 of the earbud 114a may capture one or more SpO₂ values of the patient 503.

The therapy application 216 may analyze the model of the ear canal (e.g., by comparing the model to one or more stored models of the patient 401 in the user profiles 222) to detect a change in the geometry of the ear canal (e.g., to determine the model of the ear canal is similar to a model of the ear canal while using the RPT device 106 and/or mask 108, to determine the model of the ear canal is associated with improved respiration, etc.). Similarly, the therapy application 216 may compare the SpO₂ values to one or more stored SpO₂ values in the user profile 222 for patient 503, and determine the SpO₂ values are greater than the stored SpO₂ values in the user profile 222 (and/or match stored SpO₂ values associated with the use of RPT device 106 and/or mask 108 in the user profile 222). The therapy application 216 may determine the use of the RPT device 106 and/or mask 108 by the patient 503 based on the detected change in the geometry of the ear canal, the analysis of the captured SpO₂ values, and the analysis of the soundwave 604.

In some embodiments, the analysis of the soundwave 604 includes determining an error or other problem with the mask 108. For example, the soundwave 604 may reflect an air leak in the mask 108. As such, the therapy application 216 may generate a recommendation, e.g., to replace the mask 108 and/or one or more components thereof. The type of mask 108 may be determined based on the user profile 222 and/or by detecting the type of the mask 108 (e.g., based on processing sounds made by masks 108).

For example, the therapy application 216 may determine that the soundwave 604 is associated with faulty cushions in the mask 108. In some embodiments, the therapy application 216 may transmit an instruction to the smartphone of the patient 401 to order replacement cushions without requiring user input. As another example, the therapy application 216 may determine the mask 108 is relatively new, and the cushions should not be faulty. Therefore, the therapy application 216 may determine the patient 503 is not wearing the mask 108 properly. As such, the therapy application 216 may cause the instruction manual for the mask 108 to be presented on the external device 104 (e.g., a smartphone) of the patient 503. In some embodiments, one or more pages of the instruction manual associated with properly wearing the mask 108 are presented to the user. Doing so allows the user to adjust the mask 108 so it fits properly. Over time, the therapy application 216 may determine that the user corrected the mask fit, e.g., based on absence of sounds similar to soundwave 604, improved SpO₂ values, etc. Embodiments are not limited in these contexts.

FIG. 7 is a schematic 700 depicting the human auditory system. As shown, an earbud 114a is worn in on the right ear of a patient. Earbud 114b is pictured without picturing the left ear for the sake of clarity.

As shown, one or more sounds associated with one or more soundwaves 702 are emitted from within the body of the patient. The microphone arrays 208 of the earbud 114a and/or earbud 114b may detect the soundwaves 702 and provide data associated with the detection of the soundwaves 702 to the therapy application 216. At least a portion of the soundwaves 702 are received by the microphone arrays 208 via the ear canal 704. The therapy application 216 may determine a location of a source of the soundwaves 702 as described herein.

The therapy application 216 may further generate a model of the ear canal 704 based at least in part on the soundwave 702 as described herein. The models 302, 304 are examples of models of the ear canal 704 generated by the therapy application 216.

The therapy application 216 may analyze the model of the ear canal 704 to detect a change in the geometry of the ear canal 704, e.g., by comparing the model of the ear canal 704 to one or more stored models of the ear canal 704 in the user profiles 222. For example, the change may be based on the total volume of the ear canal 704, the shape of the ear canal 704, distances between two or more locations of the ear canal, etc.

In some embodiments, based on detecting the change in the ear canal 704, the therapy application 216 may determine the patient is wearing or otherwise using a therapy device. In some embodiments, the therapy application 216 may further consider the oxygen saturation values captured by the pulse oximeter 214 associated with each model was generated to determine the patient is wearing or otherwise using a therapy device.

As stated, in some embodiments, the therapy application 216 may further process the soundwave 702 to determine the therapy device is being used. For example, if the soundwave 702 matches the stored sound of a therapy device in the device profiles 224, the therapy application 216 may determine the specific type of therapy device being used by the patient.

The therapy application 216 may further identify a pathology based on the soundwaves 702 and/or the location of the soundwaves 702, and recommend or effect a therapy or treatment for the pathology. For example, the therapy application 216 may determine that the pathology is an airway obstruction, and may identify other attributes of the airway obstruction (e.g., the type of the obstruction, whether the obstruction is partial, complete, etc.). The therapy application 216 may then generate a recommended a treatment or therapy 220 for the type of obstruction. For example, the therapy application 216 may determine to modify one or more operating parameters of the RPT device 106 to cause the RPT device 106 to deliver therapy to the patient. The therapy application 216 may further transmit an indication of the detected device, pathology, therapy 220, and/or any attributes thereof to other devices in the system 100.

FIG. 8 is a schematic 800 illustrating an example of using ear-worn devices such as earbuds to detect airway obstructions and other pathologies, in accordance with one embodiment. FIG. 8, which is not to scale, depicts a patient 802 wearing earbuds 114a, 114b. As shown, earbud 114a includes microphone arrays 208-1, 208-2, and 208-N, while earbud 114b includes microphone arrays 208-3, 208-4, and 208-M, where “N” and “M” are any positive integer (and “N” and “M” may be the same or different integers).

As shown, a soundwave 804 is emitted from the patient 802. Because the distances between each microphone array 208 in each earbud 114a, 114b, are known (e.g., fixed), each earbud 114a, 114b can independently determine the location of the origin of the soundwave 804, e.g., using triangulation, trilateration, single or multiple microphone acoustic impedance measurements, impulse/frequency response function measurements, and/or beamforming. For example, the soundwave 804 and each microphone array 208 may be a point in space to facilitate the generation of triangles to compute the location the soundwave 804 originated.

Similarly, the earbuds 114a, 114b may collectively determine the location of the origin of the soundwave 804. For example, earbud 114b may provide information describing the soundwave 804 detected by microphone arrays 208-3, 208-4, and 208-M to the earbud 114a. The therapy application 216 may then use the information from the microphone arrays 208 of both earbuds 114a, 114b to compute the location of the origin of the soundwave 804.

Furthermore, as described above, the therapy application 216 may use the determined location and any attributes of the soundwave 804, the patient 802, the ear canal, and/or the airway to determine a device associated with the soundwave 804, e.g., RPT device 106, mask 108, etc. The therapy application 216 may further generate a recommendation associated with the determined device. In some embodiments, the therapy application 216 implements the recommendation e.g., by causing the RPT device 106 to change one or more parameters of operation, etc. As another example, notifications or other instructions may be sent by the therapy application 216 to other devices in the system 100, e.g., the external devices 104, other wearables 110, RPT devices 106, masks 108, etc. Embodiments are not limited in these contexts.

Furthermore, as described above, the therapy application 216 may use the determined location and any attributes of the soundwave 804, the detected device, the patient 802, the ear canal, and/or the airway to determine a pathology associated with the soundwave 804, e.g., an OSA event, etc. The therapy application 216 may further determine a therapy 220 associated with the determined pathology. In some embodiments, the therapy application 216 implements the therapy 220, e.g., by causing the RPT device 106 to change one or more parameters of operation, etc. Embodiments are not limited in these contexts.

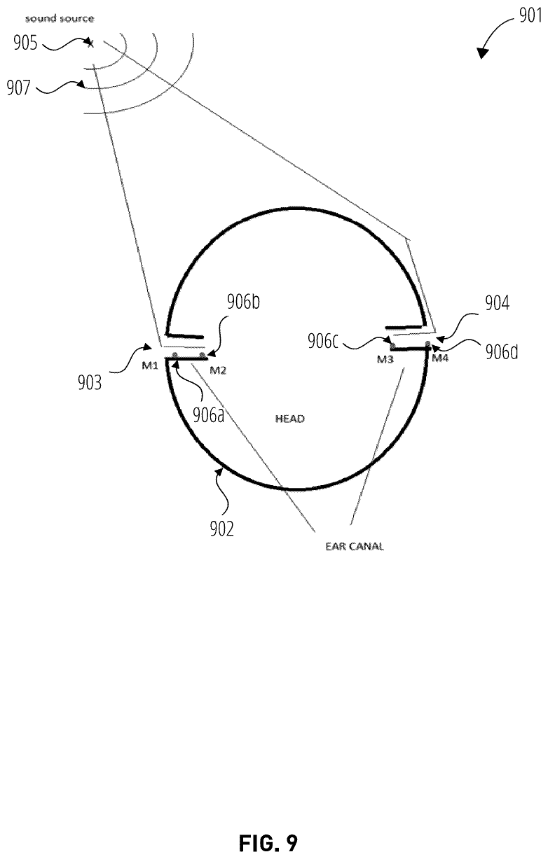

FIG. 9 is a schematic 901 illustrating an example of using ear-worn devices such as earbuds to detect therapy devices, airway obstructions, and/or other pathologies, in accordance with one embodiment. FIG. 9, which is not to scale, depicts the ear canals 903, 904 of a human head 902. Furthermore, two microphones (or microphone arrays) of an earbud (not pictured) are depicted in the ear canal of the person. For example, microphones 906a and 906b (denoted by M1 and M2, respectively) may be included in a first earbud (not pictured) located at or near ear canal 903. Similarly microphones 906c and 906d (denoted by M3, and M4, respectively) may be included a second earbud (not pictured) located at or near ear canal 904. The microphones 906a-906d are representative of one or microphones and/or microphone arrays. For example, each microphone microphones 906a-906d may be representative of a respective microphone array 208. In some embodiments, to determine the location of a sound emitted from a sound source 905, any two or more of the microphones 906a-906d may define a microphone array. Embodiments are not limited in these contexts.

As stated, the distances between any two of microphones 906a-906d may be known or otherwise determined. As shown, as one or more soundwaves 907 generated at sound source 905 moves through space, portions of the soundwaves 907 may enter each ear canal 903, 904. Therefore, portions of the soundwave 410 may be detected by the microphones 906a-906d at different times. For example, microphone 906a may detect soundwaves 907 prior to the time microphone 906b detects soundwaves 907. Similarly, microphone 906d may detect soundwaves 907 prior to microphone 906c.

Therefore, the phase relationship between the soundwaves 907 detected at each microphone 906a-906d may be used to determine the location of the sound source 905. For example, the therapy application 216 of an earbud may determine the phase shift of soundwave 907 between microphone 906a and microphone 906b, while the therapy application 216 of the other earbud in the pair may determine the phase shift of soundwaves 907 between microphone 906c and microphone 906d. As another example, one earbud may determine the phase shift of soundwaves 907 between microphone 906a and microphone 906d. Embodiments are not limited in these contexts, as the phase shift may be determined between any two or more of the microphones 906a-906d. The therapy application 216 may then compare or otherwise use the phase shifts to triangulate the location of the sound source 905, e.g., using triangulation, trilateration, time difference of arrival (TDOA), etc. For example, by converting phase shifts to time differences, the TDOA between multiple (e.g., two or more of) microphones 906a-906d may be computed to determine the location of the sound source 905.

The therapy application 216 may determine that the soundwaves 907 are associated with a therapy device used or worn by the patient based at least in part on the determined location of the sound source 905. For example, by determining the sound source 905 is located 0.5 meters away from the patient's bed and near the patient's head, the therapy application 216 may determine that an RPT device 106 is the sound source 905. Embodiments are not limited in these contexts, as other factors may be used to determine the use of the therapy device as described elsewhere herein. For example, the therapy application 216 may further determine the soundwaves 907 are similar to stored sounds of a specific type of RPT device 106 in the device profiles 224.

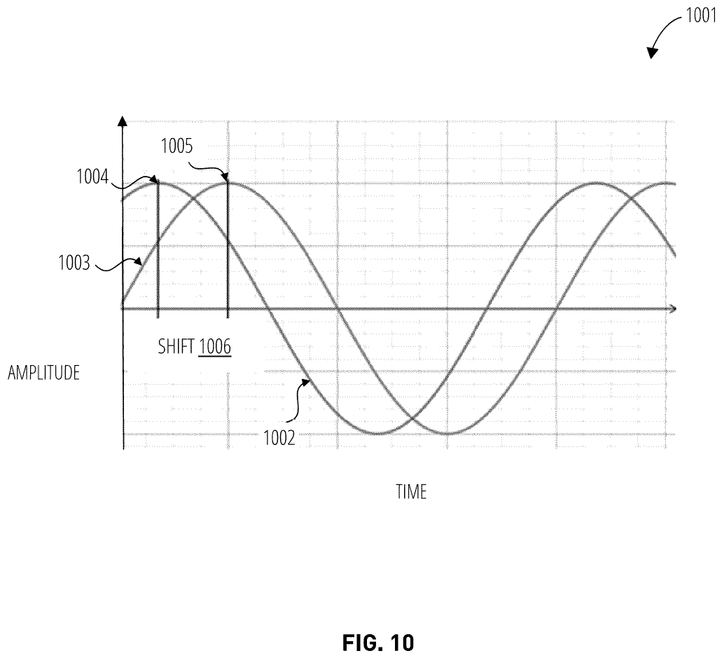

FIG. 10 is an example graph 1001 depicting two waveforms of a sound, where the x-axis corresponds to time and the y-axis corresponds to amplitude. As shown, waveforms 1002 and 1003 are depicted in the graph 1001. In one example, the waveform 1002 may be detected by microphone 906a, while waveform 1003 may be detected by microphone 906b. Although the waveforms 1002 and 1003 appear similar, they are shifted due to the different times the sound is detected by a respective microphone, which is based on the distance between the microphones and the speed of sound. Therefore, the phase shift 1006 of waveform 1002, 1003 may be determined according to any suitable technique. For example, determining the phase shift at two points 1004, 1005 may be based on cross-correlation which measures the similarity between waveforms 1002, 1003 as a function of the time-lag applied to one of them. For example, the cross-correlation may be computed using a Fast Fourier Transform (FFT). As another example, the time difference between points 1004, 1005 may be computed. The cross-correlation and/or time difference may be a time shift. The time shift may be used to compute phase shift of the waveforms 1002, 1003.

As another example, the therapy application 216 may compute phase shift 1006 by comparing the phases of waveforms 1002, 1003 in the frequency domain. For example, the therapy application 216 may compute the Fourier Transform of both waveforms 1002, 1003 to determine their frequency components. The therapy application 216 may identify the phases of the frequency components and compute the phase shift based on computing the difference of the identified phases of the frequency components. More generally, once the time shift and/or phase shifts are determined, the location a sound originated may be determined. For example, using the phase and/or time shift between microphones 906a, 906b, and 906c may be used to determine TDOA at each microphone, which may then be used to determine the location a sound originated. Embodiments are not limited in these contexts.

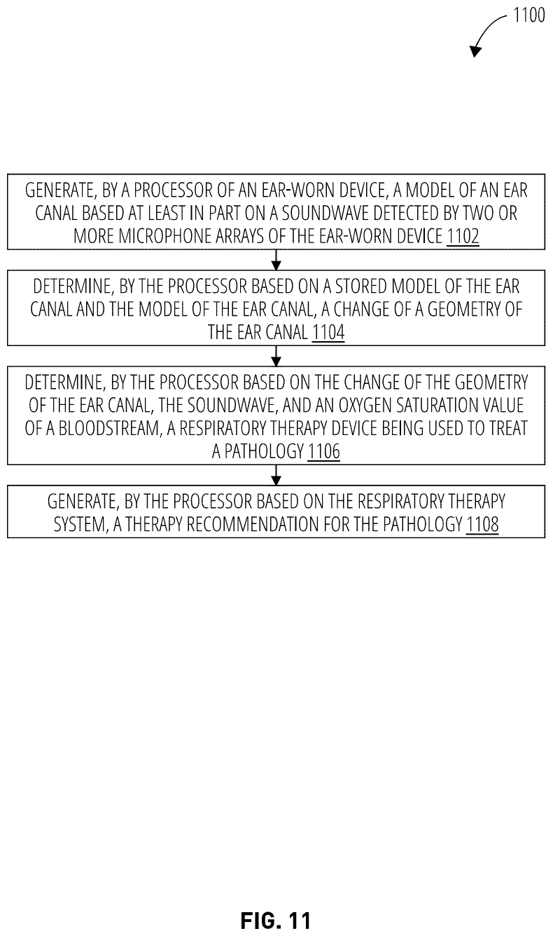

FIG. 11 illustrates an example logic flow 1100 for using ear-worn devices such as earbuds to detect therapy devices being worn or otherwise used by a human patient. Although the example logic flow 1100 depicts a particular sequence of operations, the sequence may be altered without departing from the scope of the present disclosure. For example, some of the operations depicted may be performed in parallel or in a different sequence that does not materially affect the function of the logic flow 1100. In other examples, different components of an example device or system that implements the logic flow 1100 may perform functions at substantially the same time or in a specific sequence.

According to some examples, the logic flow 1100 includes generating, by a processor of an ear-worn device, a model of an ear canal based at least in part on a soundwave detected by two or more microphone arrays of the ear-worn device at block 1102. For example, the therapy application 216 executing on processor 116e of earbud 114a may generate a model such as models 302, 304 of an ear canal based at least in part on a soundwave detected by two or more microphone arrays 208 of the earbud 114a.

According to some examples, the logic flow 1100 includes determining, by the processor based on a stored model of the ear canal and the model of the ear canal, a change of a geometry of the ear canal at block 1104. For example, the therapy application 216 executing on earbud 114a may determine, based on a stored model of the ear canal stored in the user profile 222 of the patient and the model of the ear canal generated at block 1102, a change of a geometry of the ear canal. For example, the change may be based on the volume of the ear canal, distances between two points in the ear canal, etc.

According to some examples, the logic flow 1100 includes determining, by the processor based on the change of the geometry of the ear canal, the soundwave, and an oxygen saturation value of a bloodstream, a respiratory therapy device being used to treat a pathology at block 1106. For example, the therapy application 216 executing on earbud 114a may determine, based on the change of the geometry of the ear canal determined at block 1104, the soundwave, and an oxygen saturation value of a bloodstream recorded by the pulse oximeter 214, a respiratory therapy device being used to treat a pathology. For example, the change in geometry may indicate the patient is experiencing improved respiration, the SpO₂ values may indicate improved respiration (e.g., based on previously recorded SpO₂ values and/or SpO₂ values associated with wearing a therapy device), and the soundwave may have characteristics of a therapy device. For example, the soundwave may have one or more characteristics that are similar to one or more characteristics of stored sounds in the device profiles 224. Therefore, the therapy application 216 may identify a therapy device such as RPT device 106 as being used by the patient.

According to some examples, the logic flow 1100 includes generating, by the processor based on the respiratory therapy device, a therapy recommendation at block 1108. For example, the therapy application 216 executing on earbud 114a may generate, based on the respiratory therapy device, a therapy recommendation. The therapy recommendation may be any type of recommendation. For example, the therapy recommendation may be associated with the detected device (e.g., the RPT device 106), the patient, a pathology (e.g., a detected pathology such as OSA, a pathology the detected device is associated with treating in the device profiles 224, and/or a pathology in the user profile 222), a therapy, and the like. For example, the therapy recommendation may include a new and/or modified treatment, ordering a new therapy device, ordering replacement parts for the therapy device, waking the patient, etc. The recommendation may be outputted via one or more of the earbuds 114a-114b, the external devices 104, other wearables 110, masks 108, and/or RPT devices 106. Embodiments are not limited in these contexts.

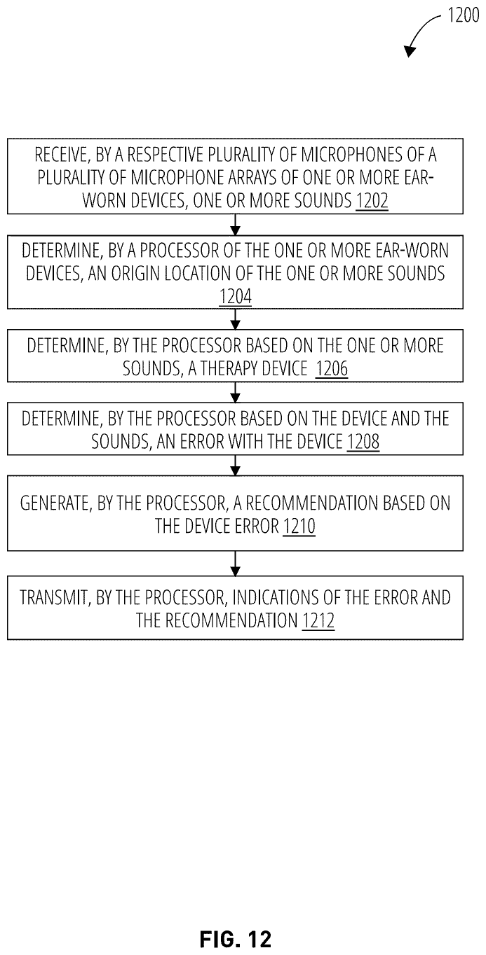

FIG. 12 illustrates an embodiment of a logic flow 1200. The logic flow 1200 represents some or all of the operations executed by one or more embodiments described herein. For example, the logic flow 1200 includes some or all of the operations performed by devices or entities in the system 100 to use ear-worn devices such as earbuds to detect therapy devices worn or otherwise used by patients. Embodiments are not limited in these contexts.

In block 1202, logic flow 1200 receives, by a respective plurality of microphones of a plurality of microphone arrays 208 of one or more ear-worn devices such as earbuds 114a, 114b, one or more sounds. The sounds may include ambient sounds, sounds generated by devices in the system 100, sounds generated by the body of the wearer of the earbuds, etc. In block 1204, logic flow 1200 determines, by a processor 116e of the one or more earbuds 114a, 114b, a location the one or more sounds originated from. For example, the therapy application 216 and/or the models 218 may process one or more of the sounds and determine the location based on triangulation, trilateration, beamforming, single or multiple microphone acoustic impedance measurements, impulse/frequency response function measurements, or any suitable technique.

In block 1206, logic flow 1200 determines, by the processor based on the one or more sounds, a therapy device associated with the sounds. For example, the therapy application 216 and/or the models 218 may process one or more of the sounds and determine the device based on the sounds, e.g., by identifying similar sounds, identifying sounds having features similar to the features of the sounds, etc. In block 1208, logic flow 1200 determines, by the processor based on the sounds and the therapy device, an error associated with the therapy device. For example, the therapy application 216 and/or the models 218 may identify a mechanical failure, air flow leaks, a configuration error, etc. In block 1210, logic flow 1200 generates, by the processor, a recommendation based on the device error determined at block 1212. For example, the therapy application 216 may determine a component of the therapy device that has failed or otherwise needs replacement.

At block 1212, the processor transmits an indication of the error and the recommendation to a device (such as any of the devices in FIG. 1) via a network such as network 112. For example, the therapy application 216 may instruct the RPT device 106 to change an attribute of the therapy delivered to the wearer. The therapy application 216 may further output an indication of the recommendation via other devices of the user, such as a smartphone, smartwatch, etc. Embodiments are not limited in these contexts.

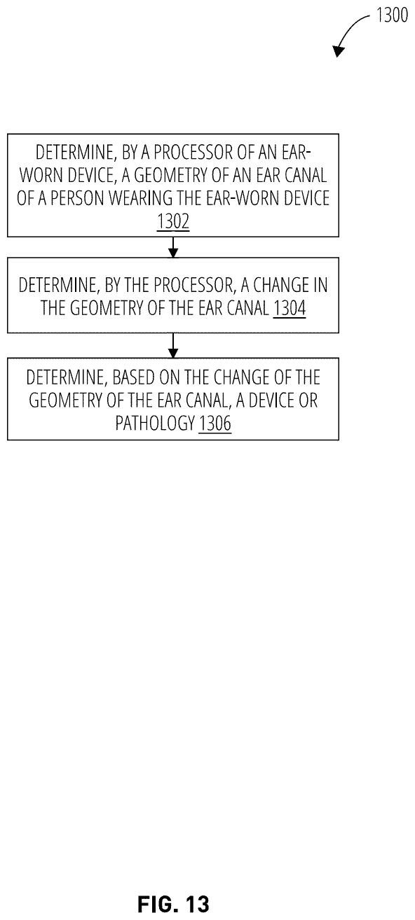

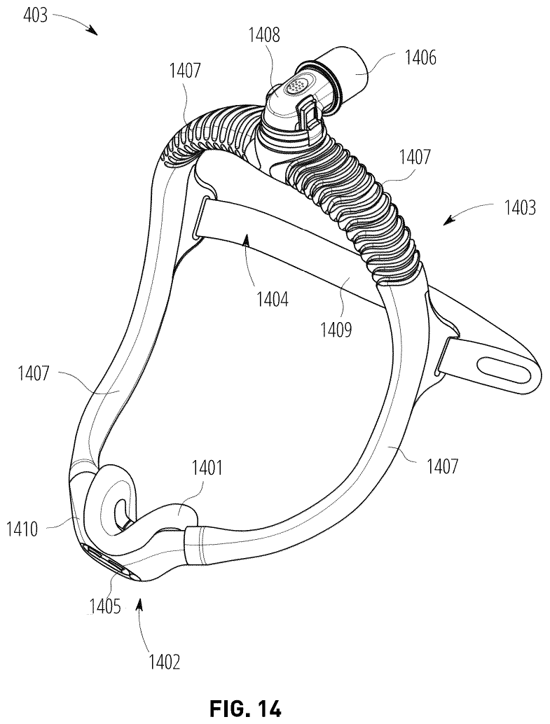

FIG. 13 illustrates an embodiment of a logic flow 1300. The logic flow 1300 represents some or all of the operations executed by one or more embodiments described herein. For example, the logic flow 1300 includes some or all of the operations performed by devices or entities in the system 100 to use ear-worn devices such as earbuds to detect airway obstructions and other pathologies. As another example, the logic flow 1300 includes some or all of the operations performed by devices or entities in the system 100 to use earbuds to detect a therapy device being worn or otherwise used by a patient. Embodiments are not limited in these contexts.

In block 1302, logic flow 1300 determines, by a processor 116e of an ear-worn device such as earbud 114a or 114b, a geometry of an ear canal of a person wearing the ear-worn device. Doing so may include generating a model of the ear canal. In block 1304, logic flow 1300 determines, by the processor, a change in the geometry of the ear canal. For example, the model generated at block 1302 may be compared to a stored model of the ear canal of the person in the user profile 222 of the person to determine one or more changes in the geometry of the ear canal.

At block 1306, the processor may determine a device worn by the person and/or a pathology of the person based on the changed geometry of the ear canal. For example, the change in geometry may reflect improved respiration by the patient in a relatively short period of time, which may cause the therapy application 216 to determine the patient is using an RPT device 106. As another example, the processor may compare the model generated at block 1302 to one or more stored models of the ear canal, e.g., one or more models in the user profiles 222 and/or the models 218. Doing so may return one or more stored models most similar to the generated model (e.g., based on shapes, abnormalities, presence of fluid, etc.). As another example, the models 218 may receive the model generated at block 1302 (and/or features thereof) and return an indication of a stored model similar to the input model. The returned models may be associated with one or more of the devices in the device profiles 224, which may in turn have an associated pathology the device is configured to treat. In some embodiments, an indication of the pathology is outputted, e.g., via the earbuds 114a-114b, the external devices 104 (e.g., the user's smartphone), the RPT devices 106 of the user, the other wearables 110 of the user (e.g., the user's smartwatch), and/or masks 108 of the user. Embodiments are not limited in these contexts.

FIG. 14 shows a patient interface 403 having conduit headgear 1404, in accordance with one embodiment. The patient interface 403 is one example of the mask 108 of FIG. 1, and therefore includes a processor 116d, memory 118d, and communications interface 120d (not pictured for clarity).

As shown, a non-invasive patient interface 403 includes a seal-forming structure 1401, a plenum chamber 1402, a positioning and stabilizing structure 1403, a vent 1405, an elbow 1408, a strap 1409, a cushion module 1410, and one embodiment of connection port 1406 for connection to air circuit 404. In some forms a functional aspect may be provided by one or more physical components. In some forms, one physical component may provide one or more functional aspects. In use the seal-forming structure 1401 is arranged to surround an entrance to the airways of the patient so as to maintain positive pressure at the entrance(s) to the airways of the patient 401. The sealed patient interface 403 is therefore suitable for delivery of positive pressure therapy, e.g., in the form of supplementary gas 1537 (e.g., oxygen).

As stated, the patient interface 403 can communicate with other devices, such as the earbuds 114a-114b and the RPT device 106., e.g., to receive instructions and modify respiratory therapy provided via the patient interface based on the instructions. The patient interface 403 is constructed and arranged to be able to provide a supply of air at a positive pressure above the ambient, for example at least 2, 4, 6, 10, or 20 cmH2O with respect to ambient.

In some embodiments, the positioning and stabilizing structure 1403 comprise one or more headgear tubes 1407 that deliver pressurized air received from a conduit forming part of the air circuit 404 from the RPT device to the patient's airways, for example through the plenum chamber 1402 and seal-forming structure 1401. In the embodiment illustrated in FIG. 14, the positioning and stabilizing structure 1403 comprises two tubes 1407 that deliver air to the plenum chamber 1402 from the air circuit 404. The tubes 1407 are configured to position and stabilize the seal-forming structure 1401 of the patient interface 403 at the appropriate part of the patient's face (for example, the nose and/or mouth) in use. This allows the conduit of air circuit 404 providing the flow of pressurized air to connect to a connection port 1406 of the patient interface in a position other than in front of the patient's face, for example on top of the patient's head.

As shown, the patient interface 403 includes a vent 1405 constructed and arranged to allow for the washout of exhaled gases, e.g., carbon dioxide. In some embodiments, the vent 1405 is configured to allow a continuous vent flow from an interior of the plenum chamber 1402 to ambient whilst the pressure within the plenum chamber is positive with respect to ambient. The vent 1405 is configured such that the vent flow rate has a magnitude sufficient to reduce rebreathing of exhaled CO2 by the patient while maintaining the therapeutic pressure in the plenum chamber in use.

Connection port 1406 allows for connection to the air circuit 404. In one or more embodiments, the patient interface 403 includes a forehead support. In one or more embodiments, the patient interface 403 includes an anti-asphyxia valve.

Air may be delivered to the patient in one of two main ways. In one example, the patient may receive the flow of pressurized air through headgear tubes 1407. This may be referred to as a “tube up” configuration and may position a connection port at the top of the patient's head. In another example, the patient may receive the flow of pressurized air through a conduit connected to the plenum chamber 1402, for example through the connection port 1406. This may be referred to a “tube down” configuration where the airflow conduit is positioned in front of the patient's face.

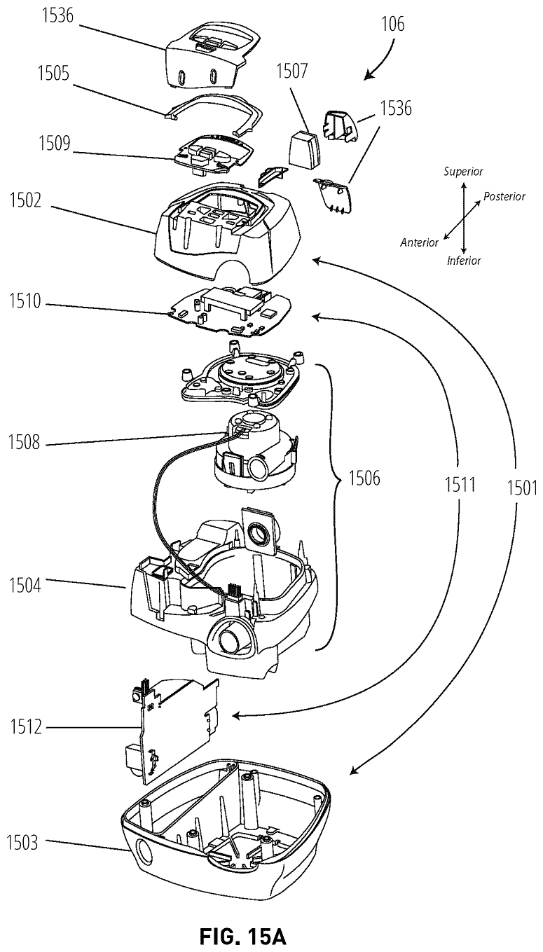

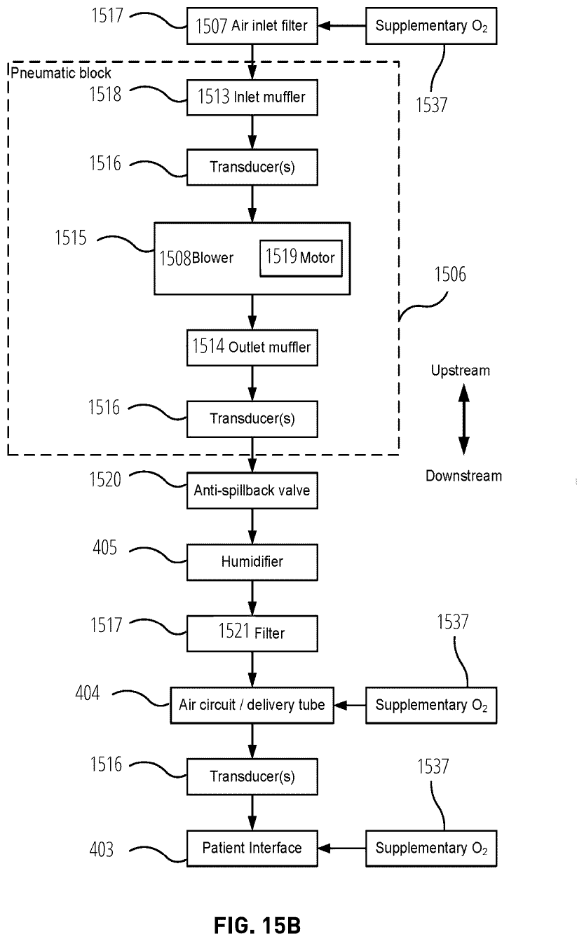

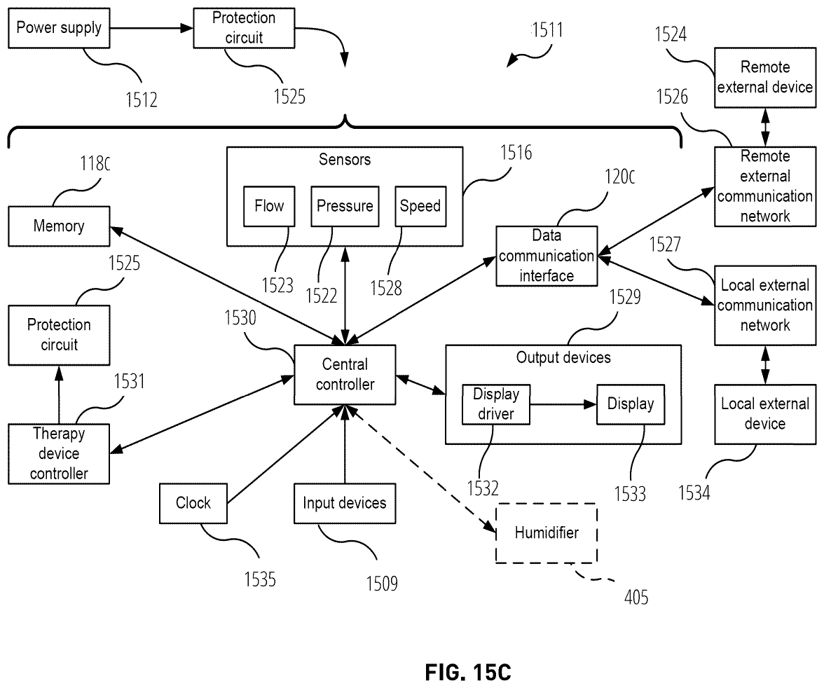

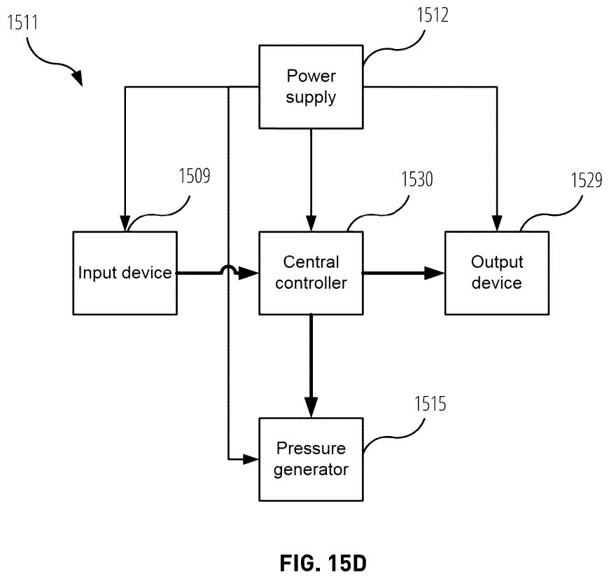

FIG. 15A shows an RPT device 106 in accordance with one embodiment. The RPT device 106 comprises mechanical, pneumatic, and/or electrical components and is configured to execute one or more algorithms, such as any of the methods, in whole or in part, described herein. The RPT device 106 may be configured to generate a flow of air for delivery to a patient's airways, such as to treat one or more of the respiratory conditions described elsewhere herein. For example, the therapy application 216 may cause the RPT device 106 to generate the flow of air to treat a pathology detected according to the techniques disclosed herein. Doing so may include the RPT device 106 modifying the delivery of therapy using one or more of the components depicted in FIG. 15A-FIG. 15D, which may include modifying any attribute thereof.

As shown in FIG. 15A, the RPT device 106 may have an external housing 1501, formed in two parts, an upper portion 1502 and a lower portion 1503. Furthermore, the external housing 1501 may include one or more panel(s) 1536. The RPT device 106 comprises a chassis 1504 that supports one or more internal components of the RPT device 106. The RPT device 106 may include a handle 1505.

One or more of the air path items may be located within a removable unitary structure which will be referred to as a pneumatic block 1506. The pneumatic block 1506 may be located within the external housing 1501. In one embodiment a pneumatic block 1506 is supported by, or formed as part of the chassis 1504.

FIG. 15B is a schematic diagram of the pneumatic path of an RPT device 106 in accordance with one or more embodiments. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

As shown in FIG. 15B, the pneumatic path of the RPT device 106 may comprise one or more air path items, e.g., an inlet air filter 1507, an inlet muffler 1513, a pressure generator 1515 capable of supplying air at positive pressure (e.g., a blower 1508), an outlet muffler 1514 and one or more transducers 1516, such as pressure sensors 1522 and flow rate sensors 1523.

The RPT device 106 may include an air filter 1517, or a plurality of air filters 1517. In the embodiment illustrated in FIG. 15B, an inlet air filter 1507 is located at the beginning of the pneumatic path upstream of a pressure generator 1515. In some embodiments, an outlet air filter 1521, for example an antibacterial filter, is located between an outlet of the pneumatic block 1506 and a patient interface 403. The RPT device 106 may include a muffler 1518, or a plurality of mufflers 1518. In one or more embodiments, an inlet muffler 1513 is located in the pneumatic path upstream of a pressure generator 1515. In one or more embodiments, an outlet muffler 1514 is located in the pneumatic path between the pressure generator 1515 and a patient interface 403.

In some embodiments, a pressure generator 1515 for producing a flow, or a supply, of air at positive pressure is a controllable blower 1508. For example, the blower 1508 may include a brushless DC motor 1519 with one or more impellers. The impellers may be located in a volute. The blower may be capable of delivering a supply of air, for example at a rate of up to about 120 liters/minute, at a positive pressure in a range from about 4 cmH2O to about 20 cmH2O, or in other forms up to about 30 cmH2O when delivering respiratory pressure therapy.

The pressure generator 1515 may be under the control of the therapy device controller 1531. In other forms, a pressure generator 1515 may be a piston-driven pump, a pressure regulator connected to a high pressure source (e.g., compressed air reservoir), or a bellows. The therapy device controller 1531 may receive instructions from the therapy application 216 and adjust the therapy based on the instruction.

In some embodiments, one or more transducers 1516 are located upstream and/or downstream of the pressure generator 1515. The one or more transducers 1516 may be constructed and arranged to generate signals representing properties of the flow of air such as a flow rate, a pressure or a temperature at that point in the pneumatic path.

In some embodiments, one or more transducers 1516 may be located proximate to the patient interface 403. In one or more embodiments, a signal from a transducer 1516 may be filtered, such as by low-pass, high-pass or band-pass filtering.

In some embodiments, a motor speed transducer 1528 is used to determine a rotational velocity of the motor 1519 and/or the blower 1508. A motor speed signal from the motor speed transducer 1528 may be provided to the therapy device controller 1531. The motor speed transducer 1528 may, for example, be a speed sensor, such as a Hall effect sensor.

As shown in FIG. 15B an anti-spill back valve 1520 is located between the humidifier 405 and the pneumatic block 1506. The anti-spill back valve is constructed and arranged to reduce the risk that water will flow upstream from the humidifier 405, for example to the motor 1519.

FIG. 15C is a schematic diagram of the electrical components 1511 of an RPT device such as RPT device 106 in accordance with one embodiment.

As shown in FIG. 15C, the RPT device 106 comprises an electrical power supply 1512, one or more input devices 1509, a central controller 1530, a therapy device controller 1531, a pressure generator 1515, one or more protection circuits 1525, memory 118c, transducers 1516, communications interface 120c and one or more output devices 1529. Electrical components 1511 may be mounted on a single Printed Circuit Board Assembly (PCBA) 1510. In an alternative form, the RPT device 106 may include more than one PCBA 1510.

The power supply 1512 may be located internal or external of the external housing 1501 of the RPT device 106. In one or more embodiments, power supply 1512 provides electrical power to the RPT device 106 only. In another embodiment, power supply 1512 provides electrical power to both RPT device 106 and humidifier 405.

In some embodiments, one or more flow rate sensors 1523 may be based on a differential pressure transducer. In one or more embodiments, a signal generated by the flow rate sensor 1523 and representing a flow rate is received by the central controller 1530. The RPT device 106 may include a clock 1535 that is connected to the central controller 1530.

In some embodiments, therapy device controller 1531 is a therapy control module that forms part of one or more algorithms executed by the central controller 1530. In one or more embodiments, therapy device controller 1531 is a dedicated motor control integrated circuit. The therapy device controller 1531 and the central controller 1530 represent the processor 116b of FIG. 1.

The one or more protection circuits 1525 may comprise an electrical protection circuit, a temperature and/or pressure safety circuit. Memory 118c may be located on the PCBA 1510. Memory 118c may be in any form. Additionally, or alternatively, RPT device 106 includes a removable form of memory 118c, for example a memory card made in accordance with the Secure Digital (SD) standard.

In one or more embodiments, the communications interface 120c is connected to the central controller 1530. Communications interface 120c may be connectable to a remote external communication network 1526 and/or a local external communication network 1527 (e.g., the network 112). The remote external communication network 1526 may be connectable to a remote external device 1524. The local external communication network 1527 may be connectable to a local external device 1534.

In one or more embodiments, remote external communication network 1526 is the Internet. The communications interface 120c may use wired communication or wireless communications to connect to the Internet. In one or more embodiments, local external communication network 1527 utilizes one or more communication standards, such as Bluetooth, a consumer infrared protocol, an I/O bus such as a universal serial bus (USB), peripheral component interconnects (PCIs), etc.

In one or more embodiments, remote external device 1524 is one or more computers, for example a cluster of networked computers. In one or more embodiments, remote external device 1524 may be virtual computers, rather than physical computers. In either case, such a remote external device 1524 may be accessible to an appropriately authorized person such as a clinician.

The local external device 1534 represents the external devices 104, which may be a personal computer, mobile phone, tablet, or remote control.

An output device 1529 may take the form of one or more of a visual, audio, and haptic unit. A visual display 1533 may be a Liquid Crystal Display (LCD) or Light Emitting Diode (LED) display. A display driver 1532 receives as an input the characters, symbols, or images intended for display on the display 1533, and converts them to commands that cause the display 1533 to display those characters, symbols, or images.

A display 1533 is configured to visually display characters, symbols, or images in response to commands received from the display driver 1532. For example, the display 1533 may be an eight-segment display, in which case the display driver 1532 converts each character or symbol, such as the figure “0”, to eight logical signals indicating whether the eight respective segments are to be activated to display a particular character or symbol.

In some embodiments, the air circuit 404 is a conduit or a tube constructed and arranged to allow, in use, a flow of air to travel between two components such as RPT device 106 and the patient interface 403. In particular, the air circuit 404 may be in fluid connection with the outlet of the pneumatic block 1506 and the patient interface. The air circuit may be referred to as an air delivery tube. In some cases there may be separate limbs of the circuit for inhalation and exhalation. In other cases a single limb is used.

In some embodiments, the air circuit 404 may comprise one or more heating elements configured to heat air in the air circuit, for example to maintain or raise the temperature of the air. The heating element may be in a form of a heated wire circuit, and may comprise one or more transducers, such as temperature sensors. In one or more embodiments, the heated wire circuit may be helically wound around the axis of the air circuit 404. The heating element may be in communication with a controller such as a central controller 1530.

As illustrated in FIG. 15D, the power supply 1512 may provide electrical power to the input devices 1509, the central controller 1530, the output device 1529, and the pressure generator 1515. The power supply 1512 may also provide electric energy to other components of the RPT device 106 (or the humidifier 405).

In one or more embodiments, an RPT device 106 includes one or more input devices 1509 in the form of buttons, switches or dials to allow a person to interact with the device. The buttons, switches or dials may be physical devices, or software devices accessible via a touch screen. The buttons, switches or dials may, in one or more embodiments, be physically connected to the external housing 1501, or may, in another form, be in wireless communication with a receiver that is in electrical connection to the central controller 1530.

In one or more embodiments, the input device 1509 may be constructed and arranged to allow a person to select a value and/or a menu option.

In one or more embodiments, the central controller 1530 is one or a plurality of processors suitable to control an RPT device 106. The central controller 1530 is shown in FIG. 15C and FIG. 15D. Suitable processors may be any of various commercially available processors. In one or more embodiments, the central controller 1530 is an application-specific integrated circuit. In another form, the central controller 1530 comprises discrete electronic components.

The central controller 1530 may be configured to receive input signal(s) from one or more transducers 1516, one or more input devices 1509, and/or the humidifier 405. The central controller 1530 may be configured to provide output signal(s) to one or more of an output device 1529, a pressure generator 1515, a therapy device controller 1531, a communications interface 120c, and/or the humidifier 405. Furthermore, central controller 1530 can receive information from or transmit information to earbuds 114a-114b.

In some embodiments, the central controller 1530 is configured to implement the one or more methodologies described herein, such as one or more algorithms which may be implemented with processor-control instructions, expressed as computer programs stored in a non-transitory computer readable storage medium, such as memory 118c. In some embodiments, the central controller 1530 may be integrated with an RPT device 106. However, in some embodiments, some methodologies may be performed by a remotely located device. For example, the remotely located device may determine control settings for a ventilator or detect respiratory related events by analysis of stored data such as from any of the sensors described herein.

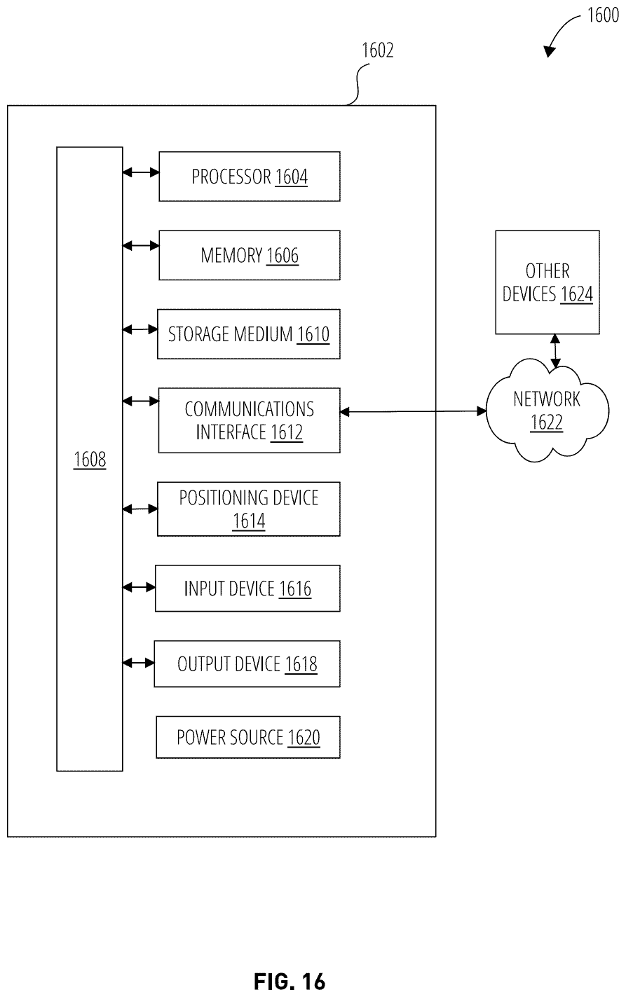

FIG. 16 illustrates an example computing system 1600 suitable for implementing various embodiments as described herein. As shown, the computing system 1600 comprises a computer 1602, which is representative of any type of physical and/or virtualized computing device. Examples of the computer 1602 include, but are not limited to, a server, workstation, laptop, mobile device, smartphone, tablet computer, mainframe, distributed computing system, compute cluster, media device, camera, gaming device, a portable digital assistant (PDA), a system-on-chip (SoC), a pager, a television, a wearable device, a virtual machine (VM), container, or any other device with processing capabilities. In one embodiment, the computer 1602 is representative of some or all of the components of the system 100. More generally, the computing system 1600 is configured to implement all systems, methods, apparatuses, media, and embodiments disclosed herein.

For example, computer 1602 may represent some or all of the components of the earbuds 114a-114b, external devices 104, RPT device 106, mask 108, other wearables 110, and/or device 504. However, all components of the computer 1602 depicted in FIG. 16 need not be included in the earbuds 114a-114b, external devices 104, RPT device 106, mask 108, other wearables 110, and/or device 504. Embodiments are not limited in these contexts.

As shown, the computer 1602 includes one or more processors 1604, one or more memories 1606, one or more non-transitory storage media 1610, one or more communications interfaces 1612, one or more positioning devices 1614, one or more input devices 1616, and one or more output devices 1618 communicably coupled via an interconnect 1608. A power source 1620, such as a power supply, battery, or any type of power source may provide power to the computer 1602.

The processor 1604 is representative of any type of processing circuit. For example, the processor 1604 may be a central processing unit (CPU), a microprocessor, a graphics processing unit (GPU), a microcontroller, an application-specific integrated circuit (ASIC), a programmable logic device (PLD), a digital signal processor (DSP), a field programmable gate array (FPGA), a state machine, a controller, gated or transistor logic, a digital signal processor, analog to digital converter, digital to analog converter, and the like.

The memory 1606 is representative of any computer readable medium to store data, code, or other information. The memory 1606 may include volatile memory, such as volatile Random Access Memory (RAM) including a cache area for the temporary storage of data. The memory 1606 may also include non-volatile memory, which can be embedded and/or may be removable. The non-volatile memory can additionally or alternatively include an electrically erasable programmable read-only memory (EEPROM), flash memory or the like. The storage medium 1610 is representative of any type of computer readable medium to store data, code, or other information. Examples of storage media 1610 include solid state drives, hard drives, Redundant Array of Independent Disks (RAID) drives, memory pools, USB storage devices, and the like.

The memory 1606 and storage medium 1610 can store any number and type of computer-executable instructions executed by the processor 1604 to implement the functions of the computer 1602 described herein. For example, the memory 1606 and/or storage medium 1610 may include the therapy application 216, the model 218, the therapies 220, the user profiles 222, and/or the device profiles 224.

The interconnect 1608 is representative of any type of circuitry to connect the components of the computer 1602. For example, the interconnect 1608 can include or represent, a system bus, a universal serial bus (USB) interface, a peripheral component interconnect (PCI), a Peripheral Component Interconnect-enhanced (PCIe), compute express link (CXL) interconnects, Universal Chiplet Interconnect Express (UCIe) interface, PCI-UCIe interconnects, an interface serial peripheral interconnects (SPIs), integrated interconnects (I2Cs), a high-speed interface connecting the processor 1604 to the memory 1606, individual electrical connections among the components, and electrical conductive traces on a motherboard common to some or all of the above-described components of the computer 1602. As discussed herein, the interconnect 1608 may operatively couple various components with one another, or in other words, electrically connects those components, either directly or indirectly—by way of intermediate component(s)—with one another.

The one or more input devices 1616 are representative of any type of input device for receiving input, such as a keypad, keyboard, touchscreen, touchpad, microphone, camera, fingerprint sensor, mouse, joystick, other pointer device, button, soft key, and the like. The one or more output devices 1618 are representative of any type of device for outputting information, such as a monitor, speaker, haptic feedback module, printer, and the like.

The computer 1602 may use the communications interface 1612 to communicate with one or more other devices 1624 via a network 1622. The communications interface 1612 allows the computer 1602 to communicate with and conduct transactions with other devices and systems, such as the other devices 1624. The communications interface 1612 may be a wired and/or a wireless interface. Thus, communications can be conducted, for example, via the wireless communications interface 1612, which can be or include a radio-frequency transceiver, a Bluetooth device, Wi-Fi device, a Near-Field Communication (NFC) device, and other wireless transceivers. In addition, a positioning device 1614 such as a Global Positioning System (GPS) device may be included for navigation and location-related data exchanges, ingoing and/or outgoing. Wi-Fi networks use radio technologies such as IEEE 802.11x (a, b, g, n, ac, ax, etc.) to provide secure, reliable, fast wireless connectivity. A Wi-Fi network connects computers to each other, to the Internet, and to wired networks (which use IEEE 802.3-related media and functions). Communications may also and/or alternatively be conducted via wired connections using the communications interface 1612, e.g., using USB, Ethernet, and other physically connected modes of data transfer. The network 1622 may be any one of, or the combination of, wired and/or wireless networks including without limitation a direct connection, a private network (e.g., an intranet), a public network (e.g., the Internet), a Personal Area Network (PAN), a Local Area Network (LAN), a Wide Area Network (WAN), a wireless network, a cellular network, and other communications networks.

The computer 1602 is configured to use the communications interface 1612 as, for example, a network interface to communicate with one or more other devices on a network such as network 1622. In this regard, the computer 1602 utilizes the wireless communications interface 1612 as an antenna operatively coupled to a transmitter and a receiver (together a “transceiver”) included with the communications interface 1612. The communications interface 1612 is configured to provide signals to and receive signals from the transmitter and receiver, respectively. The signals may include signaling information in accordance with the air interface standard of the applicable cellular system of a wireless network. In this regard, the computer 1602 may be configured to operate with one or more air interface standards, communication protocols, modulation types, and access types. By way of illustration, the computer 1602 may be configured to operate in accordance with any of a number of first, second, third, fourth, fifth-generation communication protocols and/or the like. For example, as a smartphone, the computer 1602 may be configured to operate in accordance with fourth-generation (4G) wireless communication protocols such as Long-Term Evolution (LTE), fifth-generation (5G) wireless communication protocols, Bluetooth Low Energy (BLE) communication protocols such as Bluetooth 5.0, ultra-wideband (UWB) communication protocols, and/or the like. The computer 1602 may also be configured to operate in accordance with non-cellular communication mechanisms, such as via a wireless local area network (WLAN) or other communication/data networks.

The computer 1602 may be under the control of any suitable operating system (not pictured). Example operating systems include, but are not limited to, Linux® operating systems, UNIX®, Windows® operating systems, macOS®, iOS®, Android®, and any other type of operating system.

The computer 1602 as illustrated diagrammatically represents at least one example of a possible implementation, where alternatives, additions, and modifications are possible for performing some or all of the described methods, operations, and functions. Although shown separately, in some embodiments, two or more computers 1602, systems, servers, or illustrated components may be utilized. In some implementations, the functions of one or more systems, servers, or illustrated components may be provided by a single system or server. In some embodiments, the functions of one illustrated system or server may be provided by multiple systems, servers, or computing devices, including those physically located at a central facility, those logically local, and those located as remote with respect to each other.

Aspects of the present disclosure are described herein with reference to flowchart illustrations and/or block diagrams of computer-implemented methods and computing systems according to embodiments of the disclosure. It will be understood that each block of the flowchart illustrations and/or block diagrams, and combinations of blocks in the flowchart illustrations and/or block diagrams, can be implemented by computer readable program instructions that may be provided to a processor of a computer or other programmable data processing apparatus (the term “apparatus” includes systems and computer program products). The processor may execute the computer readable program instructions thereby creating a means for implementing the actions specified in the flowchart illustrations and/or block diagrams. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the actions specified in the flowchart illustrations and/or block diagrams. In particular, the computer readable program instructions may be used to produce a computer-implemented method by executing the instructions to implement the actions specified in the flowchart illustrations and/or block diagrams.

The computer program instructions may also be stored in a computer-readable memory that can direct a computer or other programmable data processing apparatus to function in a particular manner, such that the instructions stored in the computer readable memory produce an article of manufacture including instructions, which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions, which execute on the computer or other programmable apparatus, provide steps for implementing the functions/acts specified in the flowchart and/or block diagram block or blocks. Alternatively, computer program implemented steps or acts may be combined with operator or human implemented steps or acts to carry out an embodiment.

In the flowchart illustrations and/or block diagrams disclosed herein, each block in the flowchart/diagrams may represent a module, segment, or portion of instructions, which comprises one or more executable instructions for implementing the specified logical function(s). In some implementations, the functions noted in the blocks may occur out of the order noted in the Figures. For example, two blocks shown in succession may, in fact, be executed substantially concurrently, or the blocks may sometimes be executed in the reverse order, depending upon the functionality involved.

Computer program instructions are configured to carry out operations of the present disclosure and may be or may incorporate assembler instructions, instruction-set-architecture (ISA) instructions, machine instructions, machine dependent instructions, microcode, firmware instructions, state-setting data, source code, and/or object code written in any combination of one or more programming languages.

An application program may be deployed by providing computer infrastructure operable to perform one or more embodiments disclosed herein by integrating computer readable code into a computing system thereby performing the computer-implemented methods disclosed herein.

Although various computing environments are described above, these are only examples that can be used to incorporate and use one or more embodiments. Many variations are possible.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or device that “comprises”, “has”, “includes” or “contains” one or more steps or elements possesses those one or more steps or elements, but is not limited to possessing only those one or more steps or elements. Likewise, a step of a method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described to explain the principles of one or more aspects of the disclosure and the practical application, and to enable others of ordinary skill in the art to understand one or more aspects of the disclosure for various embodiments with various modifications as are suited to the particular use contemplated.

What is claimed is:

1. A system, comprising:
a first ear-worn device; and
a second ear-worn device,
the first ear-worn device comprising:
a pulse oximeter to determine an oxygen saturation of a blood of a body;
two or more microphone arrays to receive a soundwave, wherein at least a portion of the soundwave is received via an ear canal of the body; and
a processor operable to execute one or more instructions to cause the processor to:
generate a model of the ear canal based at least in part on the soundwave;
determine, based on a stored model of the ear canal and the model of the ear canal, a change of a geometry of the ear canal;
determine, based on the change of the geometry of the ear canal, the soundwave, and the oxygen saturation, a respiratory therapy device used to treat a pathology; and
generate, based on the respiratory therapy device, a therapy recommendation.

2. The system of claim 1, the processor operable to execute one or more instructions to cause the processor to:
determine a type of a component of the respiratory therapy device; and
transmit, to an external device: (i) an indication of the type of the component, and (ii) an indication to provide instructions for using the type of the component.

3. The system of claim 1, the processor operable to execute one or more instructions to cause the processor to:
determine a rate of use of the respiratory therapy device is below a threshold;
generate, based on the determination that rate of use is below the threshold, the therapy recommendation to use the respiratory therapy device to treat the pathology; and
transmit the therapy recommendation to an external device.

4. The system of claim 1, the processor operable to execute one or more instructions to cause the processor to:
determine, based on a plurality of oxygen saturation values including the oxygen saturation determined by the pulse oximeter: (i) a first mean oxygen saturation value when the respiratory therapy device is used, and (ii) a second mean oxygen saturation value when the respiratory therapy device is not used;
determine the first mean oxygen saturation value is greater than the second mean oxygen saturation value;
generate, based on the determination that the first mean oxygen saturation value is greater than the second mean oxygen saturation value, the therapy recommendation to use the respiratory therapy device to treat the pathology; and
transmit the therapy recommendation to an external device.

5. The system of claim 1, the processor operable to execute one or more instructions to cause the processor to:
receive an indication of another soundwave received by the microphone arrays;
analyze the another soundwave to determine an airflow leak associated with a mask; and
generate the therapy recommendation based on the determined airflow leak.

6. The system of claim 5, wherein the therapy recommendation comprises replacing a cushion of the mask, the processor operable to execute one or more instructions to cause the processor to:
transmit, to an external device, an indication to order a replacement for the cushion of the mask.

7. The system of claim 6, wherein the mask is of a first mask type, the processor operable to execute one or more instructions to cause the processor to:
detect the presence of fluid in the ear canal;
determine an illness based on the presence of the fluid in the ear canal; and
based on the determined illness, output the therapy recommendation comprising use of a second mask type, wherein the second mask type is different than the first mask type.

8. The system of claim 1, wherein the instructions to generate the therapy recommendation comprise one or more instructions to cause the processor to:
determine a pressure coefficient or an airflow coefficient for the respiratory therapy device; and
transmit the pressure coefficient or the airflow coefficient to the respiratory therapy device to cause the respiratory therapy device to apply the pressure coefficient or the airflow coefficient to treat the pathology.

9. A non-transitory computer-readable storage medium, the computer-readable storage medium including instructions that when executed by a processor of an ear-worn device, cause the processor to:
generate a model of an ear canal based at least in part on a soundwave detected by two or more microphone arrays of the ear-worn device;
determine, based on a stored model of the ear canal and the model of the ear canal, a change of a geometry of the ear canal;
receive an oxygen saturation value from a pulse oximeter of the ear-worn device;
determine, based on the change of the geometry of the ear canal, the soundwave, and the oxygen saturation value, a respiratory therapy device used to treat a pathology; and
generate, based on the respiratory therapy device, a therapy recommendation.

10. The non-transitory computer-readable storage medium of claim 9, wherein the instructions further cause the processor to:
determine a type of a component of the respiratory therapy device; and
transmit, to an external device: (i) an indication of the type of the component, and (ii) an indication to provide instructions for using the type of the component.

11. The non-transitory computer-readable storage medium of claim 9, wherein the instructions further cause the processor to:
determine a rate of use of the respiratory therapy device is below a threshold;
generate, based on the determination that rate of use is below the threshold, the therapy recommendation to use the respiratory therapy device to treat the pathology; and
transmit the therapy recommendation to an external device.

12. The non-transitory computer-readable storage medium of claim 9, wherein the instructions further cause the processor to:
determine, based on a plurality of oxygen saturation values including the oxygen saturation determined by the pulse oximeter: (i) a first mean oxygen saturation value when the respiratory therapy device is used, and (ii) a second mean oxygen saturation value when the respiratory therapy device is not used;
determine the first mean oxygen saturation value is greater than the second mean oxygen saturation value;
generate, based on the determination that the first mean oxygen saturation value is greater than the second mean oxygen saturation value, the therapy recommendation to use the respiratory therapy device to treat the pathology; and
transmit the therapy recommendation to an external device.

13. The non-transitory computer-readable storage medium of claim 9, wherein the instructions further cause the processor to:
receive an indication of another soundwave received by the microphone arrays;
analyze the another soundwave to determine an airflow leak associated with a mask; and
generate the therapy recommendation based on the determined airflow leak.

14. The non-transitory computer-readable storage medium of claim 13, wherein the therapy recommendation comprises changing a type of a cushion of the mask, wherein the cushion is a first cushion type, wherein the instructions further cause the processor to:
output an indication to use of a second cushion type, wherein the second cushion type is different than the first cushion type.

15. The non-transitory computer-readable storage medium of claim 9, wherein the instructions to generate the therapy recommendation further cause the processor to:
determine a pressure coefficient or an airflow coefficient for the respiratory therapy device; and
transmit the pressure coefficient or the airflow coefficient to the respiratory therapy device to cause the respiratory therapy device to apply the pressure coefficient or the airflow coefficient to treat the pathology.

16. A method, comprising:
generating, by a processor of an ear-worn device, a model of an ear canal based at least in part on a soundwave detected by two or more microphone arrays of the ear-worn device;
determining, by the processor based on a stored model of the ear canal and the model of the ear canal, a change of a geometry of the ear canal;
determining, by the processor based on the change of the geometry of the ear canal, the soundwave, and an oxygen saturation value of a bloodstream, a respiratory therapy device being used to treat a pathology; and
generating, by the processor based on the respiratory therapy device, a therapy recommendation.

17. The method of claim 16, further comprising:
determining, by the processor, a type of a component of the respiratory therapy device; and
transmitting, by the processor to an external device: (i) an indication of the type of the component, and (ii) an indication to provide instructions for using the type of the component.

18. The method of claim 16, further comprising:
determining, by the processor, a rate of use of the respiratory therapy device is below a threshold;
generating, by the processor based on the determination that rate of use is below the threshold, the therapy recommendation to use the respiratory therapy device to treat the pathology; and
transmitting, by the processor, the therapy recommendation to an external device.

19. The method of claim 16, further comprising:
determining, by the processor based on a plurality of oxygen saturation values including the oxygen saturation value: (i) a first mean oxygen saturation value when the respiratory therapy device is used, and (ii) a second mean oxygen saturation value when the respiratory therapy device is not used;
determining, by the processor, the first mean oxygen saturation value is greater than the second mean oxygen saturation value;
generating, by the processor based on the determination that the first mean oxygen saturation value is greater than the second mean oxygen saturation value, the therapy recommendation to use the respiratory therapy device to treat the pathology; and
transmitting, by the processor, the therapy recommendation to an external device.

20. The method of claim 16, further comprising:
outputting, by the processor, the therapy recommendation on one or more of: (i) the ear-worn device, (ii) a smartphone, or (iii) a wearable device.
